(19) United States
(12) Patent Application Publication
(10) Pub. No.: US 2005/0137185 A1
(43) Pub. Date: Jun. 23, 2005

(54) Combinations of drugs for the treatment of neoplasms

(76) Inventors: Margaret S. Lee, Middleton, MA (US); Matthew James Nichols, Boston, MA (US); Yanzhen Zhang, Sudbury, MA (US); Curtis T. Keith, Boston, MA (US)

Correspondence Address: CLARK & ELBING LLP, 101 FEDERAL STREET, BOSTON, MA, 02110, US

(21) Appl. No.: 10/944,574

(22) Filed: Sep. 17, 2004

Related U.S. Application Data
(60) Provisional application No. 60/504,310, filed on Sep. 18, 2003.

(51) Int. Cl. A61K 31/55; A61K 31/5415; A61K 31/538; A61K 31/498; A61K 31/513; A61K 31/4745; A61K 31/337

(52) U.S. Cl. 514/217; 514/225.5; 514/225.8; 514/230.2; 514/250; 514/283; 514/297; 514/251; 514/449; 514/269

(57) ABSTRACT

The invention features a method for treating a patient having a cancer or other neoplasm by administering to the patient chlorpromazine or a chlorpromazine analog and an antiproliferative agent simultaneously or within 14 days of each other in amounts sufficient to treat the patient.

### CROSS-REFERENCE TO RELATED APPLICATIONS

This application claims benefit from U.S. application Ser. No. 60/504,310, filed Sep. 18, 2003, which is incorporated herein by reference.

### BACKGROUND OF THE INVENTION

The invention relates to the treatment of neoplasms such as cancer.

Cancer is a disease marked by the uncontrolled growth of abnormal cells. Cancer cells have overcome the barriers imposed on normal cells, which have a finite lifespan, to grow indefinitely. As the growth of cancer cells continue, genetic alterations may persist until the cancerous cell has manifested itself to pursue a more aggressive growth phenotype. If left untreated, metastasis, the spread of cancer cells to distant areas of the body by way of the lymph system or bloodstream, may ensue, destroying healthy tissue.

According to a recent American Cancer Society study, approximately 1,268,000 new cancer cases were expected to be diagnosed in the United States in the year 2001 alone. Lung cancer is the most common cancer-related cause of death among men and women, accounting for over 28% of all cancer-related deaths. It is the second most commonly occurring cancer among men and women; it has been estimated that there were more than 169,000 new cases of lung cancer in the U.S. in the year 2001 and accounting for 13% of all new cancer diagnoses. While the rate of lung cancer cases is declining among men in the U.S., it continues to increase among women. According to the American Cancer Society, an estimated 157,400 Americans were expected to die due to lung cancer in 2001.

Cancers that begin in the lungs are divided into two major types, non-small cell lung cancer and small cell lung cancer, depending on how the cells appear under a microscope. Non-small cell lung cancer (squamous cell carcinoma, adenocarcinoma, and large cell carcinoma) generally spreads to other organs more slowly than does small cell lung cancer. Small cell lung cancer is the less common type, accounting for about 20% of all lung cancer.

Other cancers include brain cancer, breast cancer, cervical cancer, colon cancer, gastric cancer, kidney cancer, leukemia, liver cancer, lymphoma, ovarian cancer, pancreatic cancer, prostate cancer, rectal cancer, sarcoma, skin cancer, testicular cancer, and uterine cancer. These cancers, like lung cancer, are sometimes treated with chemotherapy.

Despite the availability of numerous chemotherapeutic agents, there is still a need for treatment regimens for certain cancers, as well as a general desire for safer, more efficacious chemotherapy regimens.

### SUMMARY OF THE INVENTION

The present invention features the combination of a phenothiazine, or a phenothiazine analog or metabolite, with an antiproliferative agent for the treatment of a neoplasm.

Accordingly, in a first aspect, the invention features a method for treating a patient diagnosed with or at risk of developing a neoplasm by administering to the patient: (a) a compound having the formula (I): 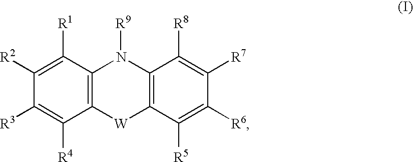
or a pharmaceutically acceptable salt thereof,

wherein R² is CF₃, halogen, OCH₃, COCH₃, CN, OCF₃, COCH₂CH₃, CO(CH₂)₂CH₃, or SCH₂CH₃;

R⁹ is selected from: 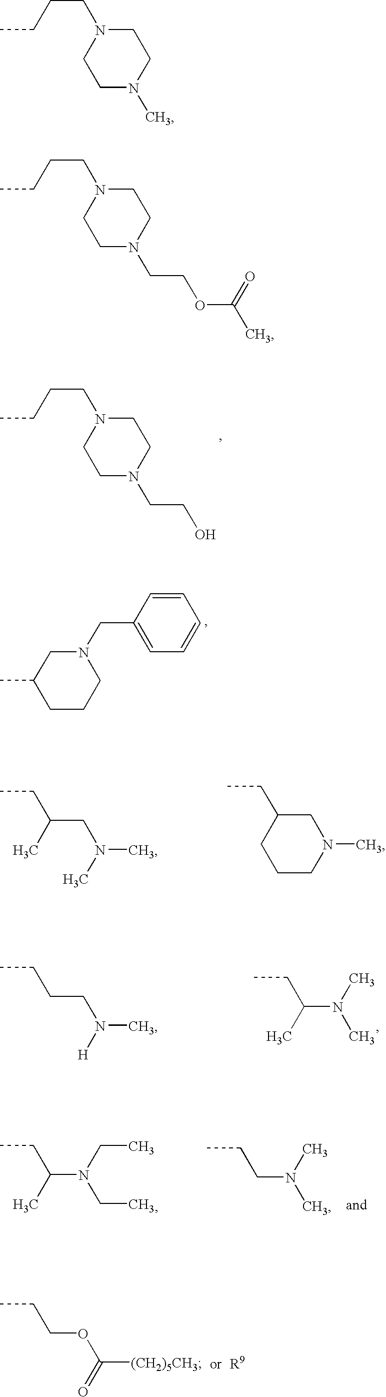
has the formula: 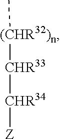
wherein n is 0 or 1, Z is NR³⁵R³⁶ or OR³⁷; each of R³², R³³, R³⁴, R¹⁵, R³⁶, and R³⁷ is, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, acyl, or C₁₋₇ heteroalkyl; or any of R³³, R³⁴, R³⁵, R³⁶, and R³⁷ can be optionally taken together with intervening carbon or non-vicinal O, S, or N atoms to form one or more five- to seven-membered rings, optionally substituted by H, halogen, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, acyl, or C₁₋₇ heteroalkyl;

each of R¹, R³, R⁴, R⁵, R⁶, R⁷, and R⁸ is independently H, OH, F, OCF₃, or OCH₃; and

W is NO,

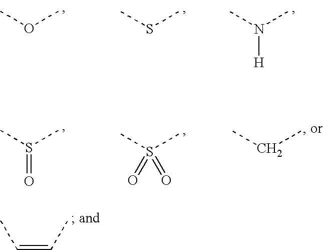

b) a Group A antiproliferative agent,

with the proviso that the method does not include administering a bis-benzimidazole compound, an endo-exonuclease inhibitor, a PRL phosphatase inhibitor, or a PTP1B inhibitor within 20 days of administering the compound of formula (I).

The compound of formula (I) and Group A antiproliferative are administered within 14 days of each other, in amounts that together are sufficient to inhibit the growth of the neoplasm. Preferably, the two compounds are administered within ten days of each other, more preferably within five days of each other, and most preferably within twenty-four hours of each other or even simultaneously.

In one embodiment, the method includes the proviso that when the compound of formula (I) is trifluoperazine, the antiproliferative agent is not doxorubicin, aclacinomycin, trifluoroacetyladriamycin-14-valerate, vinblastine, dactinomycin, colchicine, or adriamycin, and when the compound of formula (I) is chlorpromazine, the antiproliferative agent is not paclitaxel, doxorubicin, vinblastine, dactinomycin, or colchicine, and when the compound of formula (I) is thioridazine, the antiproliferative agent is not doxorubicin, vinblastine, dactinomycin, or colchicine.

Compounds of formula (I) and Group A antiproliferative agents can be administered systemically to a patient, including, without limitation, by intravenous, subcutaneous, intraperitoneal, intramuscular, inhalation, rectal, buccal, oral, or topical administration.

The invention also features a method for treating a patient diagnosed with or at risk of developing a neoplasm by administering to the patient: a) a kinesin inhibitor and b) a Group A antiproliferative agent, with the proviso that the method does not include administering a bis-benzimidazole compound, an endo-exonuclease inhibitor, a PRL phosphatase inhibitor, or a PTP1B inhibitor within 20 days of administering the kinesin inhibitor.

Kinesin inhibitors and Group A antiproliferative agents can be administered systemically to a patient, including, without limitation, by intravenous, subcutaneous, intraperitoneal, intramuscular, inhalation, rectal, buccal, oral, or topical administration.

In one embodiment, the method includes the proviso that when the kinesin inhibitor is trifluoperazine, the antiproliferative agent is not doxorubicin, aclacinomycin, trifluoroacetyladriamycin-14-valerate, vinblastine, dactinomycin, colchicine, or adriamycin, and when the kinesin inhibitor is chlorpromazine, the antiproliferative agent is not paclitaxel, doxorubicin, vinblastine, dactinomycin, or colchicine, and when the kinesin inhibitor is thioridazine, the antiproliferative agent is not doxorubicin, vinblastine, dactinomycin, or colchicine.

Neoplasms that can be treated according to any of the methods of the invention include cancers such as leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, and retinoblastoma. Preferably, the cancer being treated is lung cancer, especially lung cancer attributed to squamous cell carcinoma, adenocarinoma, or large cell carcinoma, colorectal cancer, ovarian cancer, especially ovarian adenocarcinoma, or prostate cancer.

The invention features a composition including: (a) a compound of formula (I), or a pharmaceutically acceptable salt thereof, and (b) a Group A antiproliferative agent, wherein the compound of formula (I) and the Group A antiproliferative agent are present in amounts that together are sufficient to inhibit the growth of the neoplasm when administered to a patient, and with the proviso that the composition does not include a bis-benzimidazole compound, an endo-exonuclease inhibitor, a PRL phosphatase inhibitor, or a PTP1B inhibitor.

The invention also features a composition consisting of one or more pharmaceutically acceptable excipients and a mixture of anti-neoplastic agents, wherein the mixture consists of: (a) a compound of formula (I), or a pharmaceutically acceptable salt thereof, and (b) a Group A antiproliferative agent, wherein the compound of formula (I) and the Group A antiproliferative agent are present in amounts that together are sufficient to inhibit the growth of the neoplasm when administered to a patient.

The invention further features a composition including: (a) a kinesin inhibitor, or a pharmaceutically acceptable salt thereof, and (b) a Group A antiproliferative agent, wherein the kinesin inhibitor and the Group A antiproliferative agent are present in amounts that together are sufficient to inhibit the growth of the neoplasm when administered to a patient, and with the proviso that the composition does not include a bis-benzimidazole compound, an endo-exonuclease inhibitor, a PRL phosphatase inhibitor, or a PTP1B inhibitor.

The invention also features a composition consisting of one or more pharmaceutically acceptable excipients and a mixture of anti-neoplastic agents, wherein the mixture consists of: (a) a kinesin inhibitor, or a pharmaceutically acceptable salt thereof, and (b) a Group A antiproliferative agent, wherein the kinesin inhibitor and the Group A antiproliferative agent are present in amounts that together are sufficient to inhibit the growth of the neoplasm when administered to a patient.

The compositions of the invention can be in any form, such as pill, tablet, powder, liquid etc., as further described below, such that the phenothiazine and the antiproliferative agent can be administered to a patient simultaneously or nearly simultaneously. This combination can be administered intravenously, intramuscularly, by inhalation, rectally, or by oral administration.

The invention features a kit including: (a) a compound of formula (I), or a pharmaceutically acceptable salt thereof, and (b) instructions for administering the compound of formula (I) with a Group A antiproliferative agent to a patient diagnosed with or at risk of developing a neoplasm, with the proviso that the kit does not include a bis-benzimidazole compound, an endo-exonuclease inhibitor, a PRL phosphatase inhibitor, or a PTP1B inhibitor.

The invention also features a kit including: (a) a compound of formula (I), or a pharmaceutically acceptable salt thereof, (b) a Group A antiproliferative agent, and (c) instructions for administering the compound of formula (I) and the Group A antiproliferative agent to a patient diagnosed with or at risk of developing a neoplasm, with the proviso that the kit does not include a bis-benzimidazole compound, an endo-exonuclease inhibitor, a PRL phosphatase inhibitor, or a PTP 1B inhibitor.

The kit includes the compound of formula (I) and at least one Group A antiproliferative formulated together or separately and in individual dosage amounts. When formulated separately, each may be formulated for the same or different routes of administration, including intravenous, intramuscular, inhalation, rectal, topical, or oral administration. The kit can contain one dose for a patient (single use unit dose), several doses for the same patient, or several doses for multiple patients. The kit may further include instructions for administering the compounds to treat a neoplasm, and may optionally include means for administering the unit dose, such as devices to aid administration, such as syringes, vials for reconstituting powders, or metered inhalers.

For any of the methods, compositions, and kits described herein, the compound of formula (I) or kinesin inhibitor is, desirably, acepromazine, chlorfenethazine, chlorpromazine, N-methyl chlorpromazine, cyamemazine, fluphenazine, mepazine, methotrimeprazine, methoxypromazine, norchlorpromazine, perazine, perphenazine, phenothiazine, prochlorperazine, promethazine, propiomazine, putaperazine, thiethylperazine, thiopropazate, thioridazine, trifluoperazine, or triflupromazine.

For any of the methods, compositions, and kits described herein, the Group A antiproliferative agent is, desirably, an alkylating agent (e.g., dacarbazine), an anthracycline (e.g., mitoxantrone), an anti-estrogen (e.g., bicalutamide), an anti-metabolite (e.g., floxuridine), a microtubule binding, stabilizing agent (e.g., docetaxel), microtubule binding, destabilizing agent (e.g., vinorelbine), topoisomerase inhibitor (e.g., hydroxycamptothecin (SN-38)), or a kinase inhibitor (e.g., a tyrphostin, such as AG1478). Most preferably, the agent is altretamine, carmustine, chlorambucil, cyclophosphamide, dacarbazine, ifosfamide, melphalan, mitomycin, temozolomide, doxorubicin, epirubicin, mitoxantrone, anastrazole, bicalutamide, estramustine, exemestane, flutamide, fulvestrant, tamoxifen, toremifene, capecitabine, floxuridine, fluorouracil, gemcitabine, hydroxyurea, methotrexate, gleevec, tyrphostin, docetaxel, pacilitaxel, vinblastine, vinorelbine, adjuvant/enhancing agents (celecoxib, gallium, isotretinoin, leucovorin, levamisole, pamidronate, suramin), or agents such as thalidomide, carboplatin, cisplatin, oxaliplatin, etoposide, hydroxycamptothecin, irinotecan, or topotecan. More preferably the Group A antiproliferative agent is selected from carmustine, cisplatin, etoposide, melphalan, mercaptopurine, methotrexate, mitomycin, vinblastine, paclitaxel, docetaxel, vincristine, vinorelbine, cyclophosphamide, chlorambucil, gemcitabine, capecitabine, 5-fluorouracil, fludarabine, raltitrexed, irinotecan, topotecan, doxorubicin, epirubicin, letrozole, anastrazole, formestane, exemestane, tamoxifen, toremofine, goserelin, leuporelin, bicalutamide, flutamide, nilutamide, hypericin, trastuzumab, or rituximab, or any combination thereof. Any of the antiproliferative agents listed in Table 1 can used in the methods, compositions and kits of the invention.

The invention features a method of promoting investment in a company conducting or planning in vivo studies on a composition described herein, or a company selling or planning to sell a composition described herein. The method includes the step of disseminating information about the identity, therapeutic use, toxicity, efficacy, or projected date of governmental approval of the pharmaceutical composition.

The invention also features a method of promoting investment in a company conducting or planning in vivo studies on a therapeutic method described herein. The method of promoting includes the step of disseminating information about the dosing regimen, toxicity, efficacy, or projected date of governmental approval of the therapeutic method.

As used herein “identity” refers to an identifier intended to convey the identity of a compound described herein. The identifier can be, for example, a structure, diagram, figure, chemical name, common name, tradename, formula, reference label, or any other identifier that conveys the identity of the compound to a person.

By “in vivo studies” is meant any study in which a composition of the invention is administered to a mammal, including, without limitation, non-clinical studies, e.g., to collect data concerning toxicity and efficacy, and clinical studies.

By “projected date of governmental approval” is meant any estimate of the date on which a company will receive approval from a governmental agency to sell, e.g., to patients, doctors, or hospitals, a composition or therapeutic regimen of the invention. A governmental approval includes, for example, the approval of a drug application by the Food and Drug Administration, among others.

By “Group A antiproliferative agent” is meant any antiproliferative agent, including those antiproliferative agents listed in Table 1, but excluding all bis-benzimidazole compounds, endo-exonuclease inhibitors, PRL phosphatase inhibitors, and PTP1B inhibitors.

TABLE 1
(Group A)

| | | |
|---|---|---|
| Alkylating agents | Busulfan | procarbazine |
| | dacarbazine | altretamine |
| | ifosfamide | estramustine phosphate |
| | hexamethylmelamine | mechlorethamine |
| | thiotepa | streptozocin |
| | dacarbazine | temozolomide |
| | lomustine | Semustine |
| | cyclophosphamide | cisplatin |
| | chlorambucil | |
| Platinum agents | spiroplatin | lobaplatin (Aeterna) |
| | tetraplatin | satraplatin (Johnson Matthey) |
| | ormaplatin | BBR-3464 (Hoffmann-La Roche) |
| | iproplatin | SM-11355 (Sumitomo) |
| | ZD-0473 (AnorMED) | AP-5280 (Access) |
| | oxaliplatin | |
| | carboplatin | |
| Antimetabolites | azacytidine | trimetrexate |
| | Floxuridine | deoxycoformycin |
| | 2-chlorodeoxyadenosine | pentostatin |
| | 6-mercaptopurine | hydroxyurea |
| | 6-thioguanine | decitabine (SuperGen) |
| | cytarabine | clofarabine (Bioenvision) |
| | 2-fluorodeoxy cytidine | irofulven (MGI Pharma) |
| | methotrexate | DMDC (Hoffmann-La Roche) |
| | tomudex | ethynylcytidine (Taiho) |
| | fludarabine | gemcitabine |
| | raltitrexed | capecitabine |
| Topoisomerase | amsacrine | exatecan mesylate (Daiichi) |
| inhibitors | epirubicin | quinamed (ChemGenex) |
| | etoposide | gimatecan (Sigma-Tau) |
| | teniposide or mitoxantrone | diflomotecan (Beaufour-Ipsen) |
| | 7-ethyl-10-hydroxy-camptothecin | TAS-103 (Taiho) |
| | dexrazoxanet (TopoTarget) | elsamitrucin (Spectrum) |
| | pixantrone (Novuspharma) | J-107088 (Merck & Co) |
| | rebeccamycin analogue (Exelixis) | BNP-1350 (BioNumerik) |
| | BBR-3576 (Novuspharma) | CKD-602 (Chong Kun Dang) |
| | rubitecan (SuperGen) | KW-2170 (Kyowa Hakko) |
| | irinotecan (CPT-11) | hydroxycamptothecin (SN-38) |
| | topotecan | |
| Antitumor | valrubicin | azonafide |
| antibiotics | therarubicin | anthrapyrazole |
| | idarubicin | oxantrazole |
| | rubidazone | losoxantrone |
| | plicamycin | MEN-10755 (Menarini) |
| | porfiromycin | GPX-100 (Gem Pharmaceuticals) |
| | mitoxantrone (novantrone) | Epirubicin |
| | amonafide | mitoxantrone |
| | | doxorubicin |
| Antimitotic | colchicine | E7010 (Abbott) |
| agents | vinblastine | PG-TXL (Cell Therapeutics) |
| | vindesine | IDN 5109 (Bayer) |
| | dolastatin 10 (NCI) | A 105972 (Abbott) |
| | rhizoxin (Fujisawa) | A 204197 (Abbott) |
| | mivobulin (Warner-Lambert) | LU 223651 (BASF) |
| | cemadotin (BASF) | D 24851 (ASTAMedica) |
| | RPR 109881A (Aventis) | ER-86526 (Eisai) |
| | TXD 258 (Aventis) | combretastatin A4 (BMS) |
| | epothilone B (Novartis) | isohomohalichondrin-B (PharmaMar) |
| | T 900607 (Tularik) | ZD 6126 (AstraZeneca) |
| | T 138067 (Tularik) | AZ10992 (Asahi) |
| | cryptophycin 52 (Eli Lilly) | IDN-5109 (Indena) |
| | vinflunine (Fabre) | AVLB (Prescient NeuroPharma) |
| | auristatin PE (Teikoku Hormone) | azaepothilone B (BMS) |
| | BMS 247550 (BMS) | BNP-7787 (BioNumerik) |
| | BMS 184476 (BMS) | CA-4 prodrug (OXiGENE) |
| | BMS 188797 (BMS) | dolastatin-10 (NIH) |
| | taxoprexin (Protarga) | CA-4 (OXiGENE) |
| | SB 408075 (GlaxoSmithKline) | docetaxel |
| | vinorelbine | vincristine |
| | | paclitaxel |
| Aromatase | aminoglutethimide | YM-511 (Yamanouchi) |
| inhibitors | atamestane (BioMedicines) | formestane |
| | letrozole | exemestane |
| | anastrazole | |
| Thymidylate | pemetrexed (Eli Lilly) | nolatrexed (Eximias) |
| synthase inhibitors | ZD-9331 (BTG) | CoFactor ™ (BioKeys) |
| DNA antagonists | trabectedin (PharmaMar) | edotreotide (Novartis) |
| | glufosfamide (Baxter International) | mafosfamide (Baxter International) |
| | albumin + 32P (Isotope Solutions) | apaziquone (Spectrum Pharmaceuticals) |
| | thymectacin (NewBiotics) | O6 benzyl guanine (Paligent) |
| Farnesyltransferase | arglabin (NuOncology Labs) | tipifamib (Johnson & Johnson) |
| inhibitors | lonafarnib (Schering-Plough) | perillyl alcohol (DOR BioPharma) |
| | BAY-43-9006 (Bayer) | |
| Pump inhibitors | CBT-1 (CBA Pharma) | zosuquidar trihydrochloride (Eli Lilly) |
| | tariquidar (Xenova) | biricodar dicitrate (Vertex) |
| | MS-209 (Schering AG) | |
| Histone | tacedinaline (Pfizer) | pivaloyloxymethyl butyrate (Titan) |
| acetyltransferase | SAHA (Aton Pharma) | depsipeptide (Fujisawa) |
| inhibitors | MS-275 (Schering AG) | |
| Metalloproteinase | Neovastat (Aeterna Laboratories) | CMT-3 (CollaGenex) |
| inhibitors | marimastat (British Biotech) | BMS-275291 (Celltech) |
| Ribonucleoside | gallium maltolate (Titan) | tezacitabine (Aventis) |
| reductase inhibitors | triapine (Vion) | didox (Molecules for Health) |
| TNF alpha | virulizin (Lorus Therapeutics) | revimid (Celgene) |
| agonists/antagonists | CDC-394 (Celgene) | |
| Endothelin A | atrasentan (Abbott) | YM-598 (Yamanouchi) |
| receptor antagonist | ZD-4054 (AstraZeneca) | |
| Retinoic acid | fenretinide (Johnson & Johnson) | alitretinoin (Ligand) |
| receptor agonists | LGD-1550 (Ligand) | |
| Immuno- | interferon | dexosome therapy (Anosys) |
| modulators | oncophage (Antigenics) | pentrix (Australian Cancer Technology) |
| | GMK (Progenics) | ISF-154 (Tragen) |
| | adenocarcinoma vaccine (Biomira) | cancer vaccine (Intercell) |
| | CTP-37 (AVI BioPharma) | norelin (Biostar) |
| | IRX-2 (Immuno-Rx) | BLP-25 (Biomira) |
| | PEP-005 (Peplin Biotech) | MGV (Progenics) |
| | synchrovax vaccines (CTL Immuno) | β-alethine (Dovetail) |
| | melanoma vaccine (CTL Immuno) | CLL therapy (Vasogen) |
| | p21 RAS vaccine (GemVax) | |
| Hormonal and | estrogens | dexamethasone |
| antihormonal | conjugated estrogens | prednisone |
| agents | ethinyl estradiol | methylprednisolone |
| | chlortrianisen | prednisolone |
| | idenestrol | aminoglutethimide |
| | hydroxyprogesterone caproate | leuprolide |
| | medroxyprogesterone | octreotide |
| | testosterone | mitotane |
| | testosterone propionate; fluoxymesterone | P-04 (Novogen) |
| | methyltestosterone | 2-methoxyestradiol (EntreMed) |
| | diethylstilbestrol | arzoxifene (Eli Lilly) |
| | megestrol | tamoxifen |
| | bicalutamide | toremofine |
| | flutamide | goserelin |
| | nilutamide | Leuporelin |
| | | bicalutamide |
| Photodynamic | talaporfin (Light Sciences) | Pd-bacteriopheophorbide (Yeda) |
| agents | Theralux (Theratechnologies) | lutetium texaphyrin (Pharmacyclics) |
| | motexafin gadolinium (Pharmacyclics) | hypericin |
| Kinase Inhibitors | imatinib (Novartis) | EKB-569 (Wyeth) |
| | leflunomide (Sugen/Pharmacia) | kahalide F (PharmaMar) |
| | ZD1839 (AstraZeneca) | CEP-701 (Cephalon) |
| | erlotinib (Oncogene Science) | CEP-751 (Cephalon) |
| | canertinib (Pfizer) | MLN518 (Millenium) |
| | squalamine (Genaera) | PKC412 (Novartis) |
| | SU5416 (Pharmacia) | Phenoxodiol (Novogen) |
| | SU6668 (Pharmacia ) | C225 (ImClone) |
| | ZD4190 (AstraZeneca) | rhu-Mab (Genentech) |
| | ZD6474 (AstraZeneca) | MDX-H210 (Medarex) |
| | vatalanib (Novartis) | 2C4 (Genentech) |
| | PKI166 (Novartis) | MDX-447 (Medarex) |
| | GW2016 (GlaxoSmithKline) | ABX-EGF (Abgenix) |
| | EKB-509 (Wyeth) | IMC-1C11 (ImClone) |
| | trastuzumab (Genentech) | Tyrphostins |
| | | Gefitinib (Iressa) |

Miscellaneous agents

| | |
|---|---|
| SR-27897 (CCK A inhibitor, Sanofi-Synthelabo) | ceflatonin (apoptosis promotor, ChemGenex) |
| tocladesine (cyclic AMP agonist, Ribapharm) | BCX-1777 (PNP inhibitor, BioCryst) |
| alvocidib (CDK inhibitor, Aventis) | ranpirnase (ribonuclease stimulant, Alfacell) |
| CV-247 (COX-2 inhibitor, Ivy Medical) | galarubicin (RNA synthesis inhibitor, Dong-A) |
| P54 (COX-2 inhibitor, Phytopharm) | tirapazamine (reducing agent, SRI International) |
| CapCell ™ (CYP450 stimulant, Bavarian Nordic) | N-acetylcysteine (reducing agent, Zambon) |
| GCS-100 (gal3 antagonist, GlycoGenesys) | R-flurbiprofen (NF-kappaB inhibitor, Encore) |
| G17DT immunogen (gastrin inhibitor, Aphton) | 3CPA (NF-kappaB inhibitor, Active Biotech) |
| efaproxiral (oxygenator, Allos Therapeutics) | seocalcitol (vitamin D receptor agonist, Leo) |
| PI-88 (heparanase inhibitor, Progen) | 131-I-TM-601 (DNA antagonist, TransMolecular) |
| tesmilifene (histamine antagonist, YM BioSciences) | eflornithine (ODC inhibitor, ILEX Oncology) |
| histamine (histamine H2 receptor agonist, Maxim) | minodronic acid (osteoclast inhibitor, Yamanouchi) |
| tiazofurin (IMPDH inhibitor, Ribapharm) | indisulam (p53 stimulant, Eisai) |
| cilengitide (integrin antagonist, Merck KGaA) | aplidine (PPT inhibitor, PharmaMar) |
| SR-31747 (IL-1 antagonist, Sanofi-Synthelabo) | gemtuzumab (CD33 antibody, Wyeth Ayerst) |
| CCI-779 (mTOR kinase inhibitor, Wyeth) | PG2 (hematopoiesis enhancer, Pharmagenesis) |
| exisulind (PDE V inhibitor, Cell Pathways) | Immunol ™ (triclosan oral rinse, Endo) |
| CP-461 (PDE V inhibitor, Cell Pathways) | triacetyluridine (uridine prodrug, Wellstat) |
| AG-2037 (GART inhibitor, Pfizer) | SN-4071 (sarcoma agent, Signature BioScience) |
| WX-UK1 (plasminogen activator inhibitor, Wilex) | TransMID-107 ™ (immunotoxin, KS Biomedix) |
| PBI-1402 (PMN stimulant, ProMetic LifeSciences) | PCK-3145 (apoptosis promotor, Procyon) |
| bortezomib (proteasome inhibitor, Millennium) | doranidazole (apoptosis promotor, Pola) |
| SRL-172 (T cell stimulant, SR Pharma) | CHS-828 (cytotoxic agent, Leo) |
| TLK-286 (glutathione S transferase inhibitor, Telik) | trans-retinoic acid (differentiator, NIH) |
| PT-100 (growth factor agonist, Point Therapeutics) | MX6 (apoptosis promotor, MAXIA) |
| midostaurin (PKC inhibitor, Novartis) | apomine (apoptosis promotor, ILEX Oncology) |
| bryostatin-1 (PKC stimulant, GPC Biotech) | urocidin (apoptosis promotor, Bioniche) |
| CDA-II (apoptosis promotor, Everlife) | Ro-31-7453 (apoptosis promotor, La Roche) |
| SDX-101 (apoptosis promotor, Salmedix) | brostallicin (apoptosis promotor, Pharmacia) |
| rituximab (CD20 antibody, Genentech | |

By “kinesin inhibitor” is meant a compound that inhibits by a statistically significant amount (e.g., by at least 10%, 20%, 30%, or more) the enzymatic activity of a mitotic kinesin (e.g., HsEg5). Mitotic kinesins are enzymes essential for assembly and function of the mitotic spindle and play essential roles during all phases of mitosis. Perturbation of mitotic kinesin function causes malformation or dysfunction of the mitotic spindle, frequently resulting in cell cycle arrest and cell death. Kinesin inhibitors can be identified using a variety of methods as disclosed in PCT publication WO02/057244. For example, kinesin inhibition can be identified using assays for cell cycle distribution, cell viability, morphology, activity, or by monitoring the formation of mitotic spindles. Methods for monitoring cell cycle distribution of a cell population include, for example, flow cytometry. Kinesin inhibitors include, without limitation, chlorpromazine, monasterol, terpendole E, HR22C16, and SB715992. Other mitotic kinesin inhibitors are those compounds disclosed in Hopkins et al., Biochemistry 39:2805, 2000, Hotha et al., Angew Chem. Inst. Ed. 42:2379, 2003, PCT Publication Nos. WO01/98278, WO02/057244, WO02/079169, WO02/057244, WO02/056880, WO03/050122, WO03/050064, WO03/049679, WO03/049678, WO03/049527, WO03/079973, and WO03/039460; U.S. Patent Application Publication Nos. 2002/0165240, 2003/0008888, 2003/0127621, and 2002/0143026; and U.S. Pat. No. 6,437,115, U.S. Pat. No. 6,545,004, U.S. Pat. No. 6,562,831, U.S. Pat. No. 6,569,853, and U.S. Pat. No. 6,630,479.

By “endo-exonuclease inhibitor” is meant a compound that inhibits (e.g., by at least 10%, 20%, 30%, or more) the enzymatic activity of an enzyme having endo-exonuclease activity. Such inhibitors include, but are not limited to, pentamidine, pentamidine analogs, and pentamidine metabolites.

By “phosphatase of regenerating liver inhibitor” is meant a compound that inhibits (e.g., by at least 10%, 20%, 30%, or more) the enzymatic activity of a member of the phosphatase of regenerating liver (PRL) family of tyrosine phosphatases. Members of this family include, but are not limited to, PRL-1, PRL-2, and PRL-3. Inhibitors include, but are not limited to, pentamidine, pentamidine analogs, and pentamidine metabolites.

By “protein tyrosine phosphatase 1B inhibitor” is meant a compound that inhibits (e.g., by at least 10%, 20%, 30%, or more) the enzymatic activity of protein phosphatase 1B. Inhibitors include, but are not limited to, pentamidine, pentamidine analogs, and pentamidine metabolites.

By “bis-benzimidazole compound” is meant a compound of formula (II): 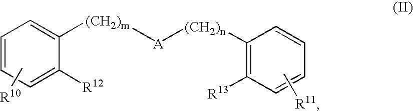
wherein A is selected from: 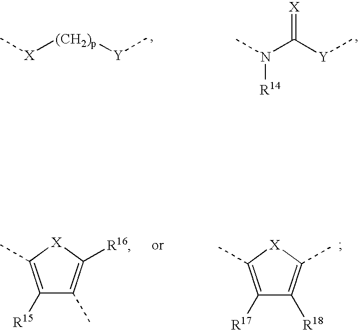
each of X and Y is, independently, O, NR¹⁹, or S; each of R¹⁴ and R¹⁹ is, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; each of R¹⁵, R¹⁶, R¹⁷, and R¹⁸ is, independently, H, halogen, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, alkoxy, arlyoxy, or C₁₋₇ heteroalkyl; p is an integer between 2 and 6, inclusive; each of m and n is, independently, an integer between 0 and 2, inclusive; each of R¹⁰ and R¹¹ is 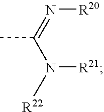
each of R²¹ and R²² is, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, acyl, or C₁₋₇ heteroalkyl; R²⁰ is H, OH, or acyl, or R²⁰ and R²¹ together represent 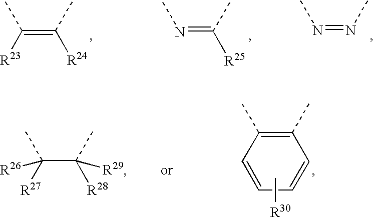
each of R²³, R²⁴, and R²⁵ is, independently, H, halogen, trifluoromethyl, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, alkoxy, arlyoxy, or C₁₋₇ heteroalkyl; each of R²⁶, R^{27 ,} R^{28 ,} and R²⁹ is, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; and R³⁰ is H, halogen, trifluoromethyl, OCF₃, NO₂, C₁₋₇ alkyl, C₂₋₇ alkenyl, C₂₋₇ alkynyl, C₂₋₆ heterocyclyl, C₆₋₁₂ aryl, C₁₋₇₋₁₄ alkaryl, C₃₋₁₀ alkheterocyclyl, alkoxy, arlyoxy, or C₁₋₇ heteroalkyl; each of R¹² and R¹³ is, independently, H, Cl, Br, OH, OCH₃, OCF₃, NO₂, and NH₂, or R¹² and R¹³ together form a single bond. Bis-benzimidazole compounds include pentamidine, propamidine, butamidine, heptamidine, nonamidine, stilbamidine, hydroxystilbamidine, diminazene, berenil, benzamidine, phenamidine, dibrompropamidine, 1,3-bis(4-amidino-2-methoxyphenoxy)propane, phenamidine, amicarbalide, 1,5-bis(4′-(N-hydroxyamidino)phenoxy)pentane, 1,3-bis(4′-(N-hydroxyamidino)phenoxy)propane, 1,3-bis(2′-methoxy-4′-(N-hydroxyamidino)phenoxy)propane, 1,4-bis(4′-(N-hydroxyamidino)phenoxy)butane, 1,5-bis(4′-(N-hydroxyamidino)phenoxy)pentane, 1,4-bis(4′-(N-hydroxyamidino)phenoxy)butane, 1,3-bis(4′-(4-hydroxyamidino)phenoxy)propane, 1,3-bis(2′-methoxy-4′-(N-hydroxyamidino)phenoxy)propane, 2,5-bis[4-amidinophenyl]furan, 2,5-bis[4-amidinophenyl]furan-bis-amidoxime, 2,5-bis[4-amidinophenyl]furan-bis-O-methylamidoxime, 2,5-bis[4-amidinophenyl]furan-bis-O-ethylamidoxime, 2,5-bis(4-amidinophenyl)furan-bis-O-4-fluorophenyl, 2,5-bis(4-amidinophenyl)furan-bis-O-4-methoxyphenyl, 2,4-bis(4-amidinophenyl)furan, 2,4-bis(4-amidinophenyl)furan-bis-O-methylamidoxime, 2,4-bis(4-amidinophenyl)furan-bis-O-4-fluorophenyl, 2,4-bis(4-amidinophenyl)furan-bis-O-4-methoxyphenyl, 2,5-bis(4-amidinophenyl) thiophene, 2,5-bis(4-amidinophenyl) thiophene-bis-O-methylamidoxime, 2,4-bis(4-amidinophenyl)thiophene, 2,4-bis(4-amidinophenyl)thiophene-bis-O-methylamidoxime, 2,8-diamidinodibenzothiophene, 2,8-bis(N-isopropylamidino)carbazole, 2,8-bis(N-hydroxyamidino)carbazole, 2,8-bis(2-imidazolinyl)dibenzothiophene, 2,8-bis(2-imidazolinyl)-5,5-dioxodibenzothiophene, 3,7-diamidinodibenzothiophene, 3,7-bis(N-isopropylamidino)dibenzothiophene, 3,7-bis(N-hydroxyamidino)dibenzothiophene, 3,7-diaminodibenzothiophene, 3,7-dibromodibenzothiophene, 3,7-dicyanodibenzothiophene, 2,8-diamidinodibenzofuran, 2,8-di(2-imidazolinyl)dibenzofuran, 2,8-di(N-isopropylamidino)dibenzofuran, 2,8-di(N-hydroxylamidino)dibenzofuran, 3,7-di(2-imidazolinyl)dibenzofuran, 3,7-di(isopropylamidino)dibenzofuran, 3,7-di(N-hydroxylamidino)dibenzofuran, 2,8-dicyanodibenzofuran, 4,4′-dibromo-2,2′-dinitrobiphenyl, 2-methoxy-2′-nitro-4,4′-dibromobiphenyl, 2-methoxy-2′-amino-4,4′-dibromobiphenyl, 3,7-dibromodibenzofuran, 3,7-dicyanodibenzofuran, 2,5-bis(5-amidino-2-benzimidazolyl)pyrrole, 2,5-bis[5-(2-imidazolinyl)-2-benzimidazolyl]pyrrole, 2,6-bis[5-(2-imidazolinyl)-2-benzimidazolyl]pyridine, 1-methyl-2,5-bis(5-amidino-2-benzimidazolyl)pyrrole, 1-methyl-2,5-bis[5-(2-imidazolyl)-2-benzimidazolyl]pyrrole, 1-methyl-2,5-bis[5-(1,4,5,6-tetrahydro-2-pyrimidinyl)-2-benzimidazolyl]pyrrole, 2,6-bis(5-amidino-2-benzimidazolyl)pyridine, 2,6-bis[5-(1,4,5,6-tetrahydro-2-pyrimidinyl)-2-benzimidazolyl]pyridine, 2,5-bis(5-amidino-2-benzimidazolyl)furan, 2,5-bis-[5-(2-imidazolinyl)-2-benzimidazolyl]furan, 2,5-bis-(5-N-isopropylamidino-2-benzimidazolyl)furan, 2,5-bis-(4-guanylphenyl)furan, 2,5-bis(4-guanylphenyl)-3,4-dimethylfuran, 2,5-bis{p-[2-(3,4,5,6-tetrahydropyrimidyl)phenyl]}furan, 2,5-bis[4-(2-imidazolinyl)phenyl]furan, 2,5 [bis-{4-(2-tetrahydropyrimidinyl)}phenyl]-3-(p-tolyloxy)furan, 2,5[bis{4-(2-imidazolinyl)}phenyl]-3-(p-tolyloxy)furan, 2,5-bis{4-[5-(N-2-aminoethylamido)benzimidazol-2-yl]phenyl}furan, 2,5-bis[4-(3a,4,5,6,7,7a-hexahydro-1H-benzimidazol-2-yl)phenyl]furan, 2,5-bis[4-(4,5,6,7-tetrahydro-1H-1,3-diazepin-2-yl)phenyl]furan, 2,5-bis(4-N,N-dimethylcarboxhydrazidephenyl)furan, 2,5-bis{4-[2-(N-2-hydroxyethyl)imidazolinyl]phenyl}furan, 2,5-bis[4-(N-isopropylamidino)phenyl]furan, 2,5-bis{4-[3-(dimethylaminopropyl)amidino]phenyl}furan, 2,5-bis{4-[N-(3-aminopropyl)amidino]phenyl}furan, 2,5-bis[2-(imidzaolinyl)phenyl]-3,4-bis(methoxymethyl)furan, 2,5-bis[4-N-(dimethylaminoethyl)guanyl]phenylfuran, 2,5-bis{4-[(N-2-hydroxyethyl)guanyl]phenyl}furan, 2,5-bis[4-N-(cyclopropylguanyl)phenyl]furan, 2,5-bis[4-(N,N-diethylaminopropyl)guanyl]phenylfuran, 2,5-bis{4-[2-(N-ethylimidazolinyl)]phenyl}furan, 2,5-bis{4-[N-(3-pentylguanyl)]}phenylfuran, 2,5-bis[4-(2-imidazolinyl)phenyl]-3-methoxyfuran, 2,5-bis[4-(N-isopropylamidino)phenyl]-3-methylfuran, bis[5-amidino-2-benzimidazolyl]methane, bis[5-(2-imidazolyl)-2-benzimidazolyl]methane, 1,2-bis[5-amidino-2-benzimidazolyl]ethane, 1,2-bis[5-(2-imidazolyl)-2-benzimidazolyl]ethane, 1,3-bis[5-amidino-2-benzimidazolyl]propane, 1,3-bis[5-(2-imidazolyl)-2-benzimidazolyl]propane, 1,4-bis[5-amidino-2-benzimidazolyl]propane, 1,4-bis[5-(2-imidazolyl)-2-benzimidazolyl]butane, 1,8-bis[5-amidino-2-benzimidazolyl]octane, trans-1,2-bis[5-amidino-2-benzimidazolyl]ethene, 1,4-bis[5-(2-imidazolyl)-2-benzimidazolyl]-1-butene, 1,4-bis[5-(2-imidazolyl)-2-benzimidazolyl]-2-butene, 1,4-bis[5-(2-imidazolyl)-2-benzimidazolyl]-1-methylbutane, 1,4-bis[5-(2-imidazolyl)-2-benzimidazolyl]-2-ethylbutane, 1,4-bis[5-(2-imidazolyl)-2-benzimidazolyl]-1-methyl-1-butene, 1,4-bis[5-(2-imidazolyl)-2-benzimidazolyl]-2,3-diethyl-2-butene, 1,4-bis[5-(2-imidazolyl)-2-benzimidazolyl]-1,3-butadiene, 1,4-bis[5-(2-imidazolyl)-2-benzimidazolyl]-2-methyl-1,3-butadiene, bis[5-(2-pyrimidyl)-2-benzimidazolyl]methane, 1,2-bis[5-(2-pyrimidyl)-2-benzimidazolyl]ethane, 1,3-bis[5-amidino-2-benzimidazolyl]propane, 1,3-bis[5-(2-pyrimidyl)-2-benzimidazolyl]propane, 1,4-bis[5-(2-pyrimidyl)-2-benzimidazolyl]butane, 1,4-bis[5-(2-pyrimidyl)-2-benzimidazolyl]-1-butene, 1,4-bis[5-(2-pyrimidyl)-2-benzimidazolyl]-2-butene, 1,4-bis[5-(2-pyrimidyl)-2-benzimidazolyl]-1-methylbutane, 1,4-bis[5-(2-pyrimidyl)-2-benzimidazolyl]-2-ethylbutane, 1,4-bis[5-(2-pyrimidyl)-2-benzimidazolyl]-1-methyl-1-butene, 1,4-bis[5-(2-pyrimidyl)-2-benzimidazolyl]-2,3-diethyl-2-butene, 1,4-bis[5-(2-pyrimidyl)-2-benzimidazolyl]-1,3-butadiene, and 1,4-bis[5-(2-pyrimidyl)-2-benzimidazolyl]-2-methyl-1,3-butadiene, 2,4-bis(4-guanylphenyl)pyrimidine, 2,4-bis(4-imidazolin-2-yl)pyrimidine, 2 ,4-bis[(tetrahydropyrimidinyl-2-yl)phenyl]pyrimidine, 2-(4-[N-i-propylguanyl]phenyl)-4-(2-methoxy-4-[N-i-propylguanyl]phenyl)pyrimidine, 4-(N-cyclopentylamidino)-1,2-phenylene diamine, 2,5-bis-[2-(5-amidino)benzimidazoyl]furan, 2,5-bis[2-{5-(2-imidazolino)}benzimidazoyl]furan, 2,5-bis[2-(5-N-isopropylamidino)benzimidazoyl]furan, 2,5-bis[2-(5-N-cyclopentylamidino)benzimidazoyl]furan, 2,5-bis[2-(5-amidino)benzimidazoyl]pyrrole, 2,5-bis[2-{5-(2-imidazolino)}benzimidazoyl]pyrrole, 2,5-bis[2-(5-N-isopropylamidino)benzimidazoyl]pyrrole, 2,5-bis[2-(5-N-cyclopentylamidino)benzimidazoyl]pyrrole, 1-methyl-2,5-bis[2-(5-amidino)benzimidazoyl]pyrrole, 2,5-bis[2-{5-(2-imidazolino)}benzimidazoyl]-1-methylpyrrole, 2,5-bis[2-(5-N-cyclopentylamidino)benzimidazoyl]-1-methylpyrrole, 2,5-bis[2-(5-N-isopropylamidino)benzimidazoyl]thiophene, 2,6-bis[2-{5-(2-imidazolino)}benzimidazoyl]pyridine, 2,6-bis[2-(5-amidino)benzimidazoyl]pyridine, 4,4′-bis[2-(5-N-isopropylamidino)benzimidazoyl]-1,2-diphenylethane, 4,4′-bis[2-(5-N-cyclopentylamidino)benzimidazoyl]-2,5-diphenylfuran, 2,5-bis[2-(5-amidino)benzimidazoyl]benzo[b]furan, 2,5-bis[2-(5-N-cyclopentylamidino)benzimidazoyl]benzo[b]furan, 2,7-bis[2-(5-N-isopropylamidino)benzimidazoyl]fluorine, 2,5-bis[4-(3-(N-morpholinopropyl)carbamoyl)phenyl]furan, 2,5-bis[4-(2-N,N-dimethylaminoethylcarbamoyl)phenyl]furan, 2,5-bis[4-(3-N,N-dimethylaminopropylcarbamoyl)phenyl]furan, 2,5-bis[4-(3-N-methyl-3-N-phenylaminopropylcarbamoyl)phenyl]furan, 2,5-bis[4-(3-N, N⁸,N¹¹-trimethylaminopropylcarbamoyl)phenyl]furan, 2,5-bis[3-amidinophenyl]furan, 2,5-bis[3-(N-isopropylamidino)amidinophenyl]furan, 2,5-bis[3[(N-(2-dimethylaminoethyl)amidino]phenylfuran, 2,5-bis[4-(N-2,2,2-trichloroethoxycarbonyl)amidinophenyl]furan, 2,5-bis[4-(N-thioethylcarbonyl) amidinophenyl]furan, 2,5-bis[4-(N-benzyloxycarbonyl)amidinophenyl]furan, 2,5-bis[4-(N-phenoxycarbonyl)amidinophenyl]furan, 2,5-bis[4-(N-(4-fluoro)-phenoxycarbonyl)amidinophenyl]furan, 2,5-bis[4-(N-(4-methoxy)phenoxycarbonyl)amidinophenyl]furan, 2,5-bis[4(1-acetoxyethoxycarbonyl)amidinophenyl]furan, and 2,5-bis[4-(N-(3-fluoro)phenoxycarbonyl)amidinophenyl]furan, or a salt of any of the above. Bis-benzimidazole compounds also include functional analogs of pentamidine, such as netropsin, distamycin, bleomycin, actinomycin, daunorubicin. Bis-benzimidazole compounds further include any compound that falls within a formula provided in any of U.S. Pat. No. 5,428,051; U.S. Pat. No. 5,521,189; U.S. Pat. No. 5,602,172; U.S. Pat. No. 5,643,935; U.S. Pat. No. 5,723,495; U.S. Pat. No. 5,843,980; U.S. Pat. No. 6,008,247; U.S. Pat. No. 6,025,398; U.S. Pat. No. 6,172,104; U.S. Pat. No. 6,214,883; and U.S. Pat. No. 6,326,395, and any compound that falls within a formula provided in any of U.S. Patent Application Publication Nos. US 2001/0044468 A1 and US 2002/0019437 A1. Bis-benzimidazole compounds include any compound identified as a pentamidine analog, or falling within a formula which includes pentamidine, provided in U.S. Pat. No. 6,569,853 and in U.S. Patent Application Publication No. 20040116407 A1.

As used herein, by the terms “cancer” or “neoplasm” or “neoplastic cells” is meant a collection of cells multiplying in an abnormal manner. Cancer growth is uncontrolled and progressive, and occurs under conditions that would not elicit, or would cause cessation of, multiplication of normal cells. The terms also encompass neoplasms, cancers, or neoplastic cells located at the original site of proliferation (“primary tumor or cancer”) and their invasion of other tissues, or organs beyond the primary site (“metastisis”).

By “inhibits the growth of a neoplasm” is meant measurably slows, stops, or reverses the growth rate of the neoplasm or neoplastic cells in vitro or in vivo. Desirably, a slowing of the growth rate is by at least 20%, 30%, 50%, or even 70%, as determined using a suitable assay for determination of cell growth rates (e.g., a cell growth assay described herein). Typically, a reversal of growth rate is accomplished by initiating or accelerating necrotic or apoptotic mechanisms of cell death in the neoplastic cells, resulting in a shrinkage of the neoplasm.

By “an amount sufficient” is meant the amount of a compound, in a combination of the invention, required to treat or prevent a neoplasm in a clinically relevant manner. A sufficient amount of an active compound used to practice the present invention for therapeutic treatment of a neoplasm varies depending upon the manner of administration, the age, body weight, and general health of the patient. Ultimately, the prescribers will decide the appropriate amount and dosage regimen. Additionally, a sufficient amount can be that amount of compound in the combination of the invention that is safe and efficacious in the treatment of a patient having, or at risk of, a neoplasm over each agent alone as determined and approved by a regulatory authority (such as the U.S. Food and Drug Administration).

The term “administration” or “administering” refers to a method of giving a compositions of the invention, by a route selected from, without limitation, inhalation, ocular administration, nasal instillation, parenteral administration, dermal administration, transdermal administration, buccal administration, rectal administration, sublingual administration, perilingual administration, nasal administration, topical administration and oral administration. Parenteral administration includes intravenous, intraperitoneal, subcutaneous, and intramuscular administration. The preferred method of administration can vary depending on various factors, e.g., the components of the pharmaceutical composition, site of the potential or actual disease and severity of disease.

In the generic descriptions of compounds of this invention, the number of atoms of a particular type in a substituent group is generally given as a range, e.g., an alkyl group containing from 1 to 7 carbon atoms or C₁₋₇ alkyl. Reference to such a range is intended to include specific references to groups having each of the integer number of atoms within the specified range. For example, an alkyl group from 1 to 7 carbon atoms includes each of C₁, C₂, C₃, C₄, C₅, C₆, and C₇. A C₁₋₇ heteroalkyl, for example, includes from 1 to 6 carbon atoms in addition to one or more heteroatoms. Other numbers of atoms and other types of atoms may be indicated in a similar manner.

As used herein, the terms “alkyl” and the prefix “alk-” are inclusive of both straight chain and branched chain groups and of cyclic groups, i.e., cycloalkyl. Cyclic groups can be monocyclic or polycyclic and preferably have from 3 to 6 ring carbon atoms, inclusive. Exemplary cyclic groups include cyclopropyl, cyclobutyl, cyclopentyl, and cyclohexyl groups. The alkyl group may be substituted or unsubstituted. Exemplary substituents include alkoxy, aryloxy, sulfhydryl, alkylthio, arylthio, halogen, hydroxyl, fluoroalkyl, perfluoralkyl, amino, aminoalkyl, disubstituted amino, quaternary amino, hydroxyalkyl, carboxyalkyl, and carboxyl groups. Exemplary alkyls include, without limitation, methyl; ethyl; n-propyl; isopropyl; cyclopropyl; cyclopropylmethyl; cyclopropylethyl; n-butyl; iso-butyl; sec-butyl; tert-butyl; cyclobutyl; cyclobutylmethyl; cyclobutylethyl; n-pentyl; cyclopentyl; cyclopentylmethyl; cyclopentylethyl; 1-methylbutyl; 2-methylbutyl; 3-methylbutyl; 2,2-dimethylpropyl; 1-ethylpropyl; 1,1-dimethylpropyl; 1,2-dimethylpropyl; 1-methylpentyl; 2-methylpentyl; 3-methylpentyl; 4-methylpentyl; 1,1-dimethylbutyl; 1,2-dimethylbutyl; 1,3-dimethylbutyl; 2,2-dimethylbutyl; 2,3-dimethylbutyl; 3,3-dimethylbutyl; 1-ethylbutyl; 2-ethylbutyl; 1,1,2-trimethylpropyl; 1,2,2-trimethylpropyl; 1-ethyl-1-methylpropyl; 1-ethyl-2-methylpropyl; and cyclohexyl.

By “alkenyl” is meant a branched or unbranched hydrocarbon group containing one or more double bonds. An alkenyl may optionally include monocyclic or polycyclic rings, in which each ring desirably has from three to six members. The alkenyl group may be substituted or unsubstituted. Exemplary substituents include alkoxy, aryloxy, sulfhydryl, alkylthio, arylthio, halogen, hydroxyl, fluoroalkyl, perfluoralkyl, amino, aminoalkyl, disubstituted amino, quaternary amino, hydroxyalkyl, carboxyalkyl, and carboxyl groups. Exemplary alkenyls include, without limitation, vinyl; allyl; 2-cyclopropyl-1-ethenyl; 1-propenyl; 1-butenyl; 2-butenyl; 3-butenyl; 2-methyl-1-propenyl; 2-methyl-2-propenyl; 1-pentenyl; 2-pentenyl; 3-pentenyl; 4-pentenyl; 3-methyl-1-butenyl; 3-methyl-2-butenyl; 3-methyl-3-butenyl; 2-methyl-1-butenyl; 2-methyl-2-butenyl; 2-methyl-3-butenyl; 2-ethyl-2-propenyl; 1-methyl-1-butenyl; 1-methyl-2-butenyl; 1-methyl-3-butenyl; 2-methyl-2-pentenyl; 3-methyl-2-pentenyl; 4-methyl-2-pentenyl; 2-methyl-3-pentenyl; 3-methyl-3-pentenyl; 4-methyl-3-pentenyl; 2-methyl-4-pentenyl; 3-methyl-4-pentenyl; 1,2-dimethyl-1-propenyl; 1,2-dimethyl-1-butenyl; 1,3-dimethyl-1-butenyl; 1,2-dimethyl-2-butenyl; 1,1-dimethyl-2-butenyl; 2,3-dimethyl-2-butenyl; 2,3-dimethyl-3-butenyl; 1,3-dimethyl-3-butenyl; 1,1-dimethyl-3-butenyl and 2,2-dimethyl-3-butenyl.

By “alkynyl” is meant a branched or unbranched hydrocarbon group containing one or more triple bonds. An alkynyl may optionally include monocyclic, bicyclic, or tricyclic rings, in which each ring desirably has five or six members. The alkynyl group may be substituted or unsubstituted. Exemplary substituents include alkoxy, aryloxy, sulfhydryl, alkylthio, arylthio, halogen, hydroxy, fluoroalkyl, perfluoralkyl, amino, aminoalkyl, disubstituted amino, quaternary amino, hydroxyalkyl, carboxyalkyl, and carboxyl groups. Exemplary alkynyls include, without limitation, ethynyl, 1-propynyl, 2-propynyl, 1-butynyl, 2-butynyl, 3-butynyl, 1-pentynyl, 2-pentynyl, 3-pentynyl, 4-pentynyl, 5-hexene-1-ynyl, 2-hexynyl, 3-hexynyl, 4-hexynyl, 5-hexynyl; 1-methyl-2-propynyl; 1-methyl-2-butynyl; 1-methyl-3-butynyl; 2-methyl-3-butynyl; 1,2-dimethyl-3-butynyl; 2,2-dimethyl-3-butynyl; 1-methyl-2-pentynyl; 2-methyl-3-pentynyl; 1-methyl-4-pentynyl; 2-methyl-4-pentynyl; and 3-methyl-4-pentynyl.

By “C₂₋₆ heterocyclyl” is meant a stable 5- to 7-membered monocyclic or 7- to 14-membered bicyclic heterocyclic ring which is saturated partially unsaturated or unsaturated (aromatic), and which consists of 2 to 6 carbon atoms and 1, 2, 3 or 4 heteroatoms independently selected from the group consisting of N, O, and S and including any bicyclic group in which any of the above-defined heterocyclic rings is fused to a benzene ring. The heterocyclyl group may be substituted or unsubstituted. Exemplary substituents include alkoxy, aryloxy, sulfhydryl, alkylthio, arylthio, halogen, hydroxy, fluoroalkyl, perfluoralkyl, amino, aminoalkyl, disubstituted amino, quaternary amino, hydroxyalkyl, carboxyalkyl, and carboxyl groups. The nitrogen and sulfur heteroatoms may optionally be oxidized. The heterocyclic ring may be covalently attached via any heteroatom or carbon atom which results in a stable structure, e.g., an imidazolinyl ring may be linked at either of the ring-carbon atom positions or at the nitrogen atom. A nitrogen atom in the heterocycle may optionally be quaternized. Preferably when the total number of S and O atoms in the heterocycle exceeds 1, then these heteroatoms are not adjacent to one another. Heterocycles include, without limitation, 1H-indazole, 2-pyrrolidonyl, 2H,6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazalonyl, carbazolyl, 4aH-carbazolyl, b-carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, 4H-quinolizinyl, quinoxalinyl, quinuclidinyl, carbolinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, xanthenyl. Preferred 5 to 10 membered heterocycles include, but are not limited to, pyridinyl, pyrimidinyl, triazinyl, furanyl, thienyl, thiazolyl, pyrrolyl, pyrazolyl, imidazolyl, oxazolyl, isoxazolyl, tetrazolyl, benzofuranyl, benzothiofuranyl, indolyl, benzimidazolyl, 1H-indazolyl, oxazolidinyl, isoxazolidinyl, benzotriazolyl, benzisoxazolyl, oxindolyl, benzoxazolinyl, quinolinyl, and isoquinolinyl. Preferred 5 to 6 membered heterocycles include, without limitation, pyridinyl, pyrimidinyl, triazinyl, furanyl, thienyl, thiazolyl, pyrrolyl, piperazinyl, piperidinyl, pyrazolyl, imidazolyl, oxazolyl, isoxazolyl, and tetrazolyl.

By “C₆₋₁₂ aryl” is meant an aromatic group having a ring system comprised of carbon atoms with conjugated π electrons (e.g., phenyl). The aryl group has from 6 to 12 carbon atoms. Aryl groups may optionally include monocyclic, bicyclic, or tricyclic rings, in which each ring desirably has five or six members. The aryl group may be substituted or unsubstituted. Exemplary subsituents include alkyl, hydroxy, alkoxy, aryloxy, sulfhydryl, alkylthio, arylthio, halogen, fluoroalkyl, carboxyl, hydroxyalkyl, carboxyalkyl, amino, aminoalkyl, monosubstituted amino, disubstituted amino, and quaternary amino groups.

By “C₇₋₁₄ alkaryl” is meant an alkyl substituted by an aryl group (e.g., benzyl, phenethyl, or 3,4-dichlorophenethyl) having from 7 to 14 carbon atoms.

By “C₃₋₁₀ alkheterocyclyl” is meant an alkyl substituted heterocyclic group having from 7 to 14 carbon atoms in addition to one or more heteroatoms (e.g., 3-furanylmethyl, 2-furanylmethyl, 3-tetrahydrofuranylmethyl, or 2-tetrahydrofuranylmethyl).

By “heteroalkyl” is meant a branched or unbranched alkyl, alkenyl, or alkynyl group having a number of carbon atoms, e.g., from 1 to 7 carbon atoms, in addition to 1, 2, 3 or 4 heteroatoms independently selected from the group consisting of N, O, S, and P. Heteroalkyls include, without limitation, tertiary amines, secondary amines, ethers, thioethers, amides, thioamides, carbamates, thiocarbamates, hydrazones, imines, phosphodiesters, phosphoramidates, sulfonamides, and disulfides. A heteroalkyl may optionally include monocyclic, bicyclic, or tricyclic rings, in which each ring desirably has three to six members. The heteroalkyl group may be substituted or unsubstituted. Exemplary substituents include alkoxy, aryloxy, sulfhydryl, alkylthio, arylthio, halogen, hydroxyl, fluoroalkyl, perfluoralkyl, amino, aminoalkyl, disubstituted amino, quaternary amino, hydroxyalkyl, hydroxyalkyl, carboxyalkyl, and carboxyl groups.

By “acyl” is meant a chemical moiety with the formula R—C(O)—, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₇ heteroalkyl.

By “halogen” is meant bromine, chlorine, iodine, or fluorine.

By “fluoroalkyl” is meant an alkyl group that is substituted with a fluorine.

By “perfluoroalkyl” is meant an alkyl group consisting of only carbon and fluorine atoms.

By “carboxyalkyl” is meant a chemical moiety with the formula -(R)-COOH, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “hydroxyalkyl” is meant a chemical moiety with the formula -(R)-OH, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₇ heteroalkyl.

By “alkoxy” is meant a chemical substituent of the formula —OR, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₇ heteroalkyl.

By “aryloxy” is meant a chemical substituent of the formula —OR, wherein R is a C₆₋₁₂ aryl group.

By “alkylthio” is meant a chemical substituent of the formula —SR, wherein R is selected from C₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “arylthio” is meant a chemical substituent of the formula —SR, wherein R is a C₆₋₁₂ aryl group.

By “quaternary amino” is meant a chemical substituent of the formula —(R)—N(R′)(R″)(R′″)⁺, wherein R, R′, R″, and R′″ are each independently an alkyl, alkenyl, alkynyl, or aryl group. R may be an alkyl group linking the quaternary amino nitrogen atom, as a substituent, to another moiety. The nitrogen atom, N, is covalently attached to four carbon atoms of alkyl and/or aryl groups, resulting in a positive charge at the nitrogen atom.

Compounds useful in the invention include those described herein in any of their pharmaceutically acceptable forms, including isomers such as diastereomers and enantiomers, salts, solvates, and polymorphs, thereof, as well as racemic mixtures of the compounds described herein.

Other features and advantages of the invention will be apparent from the following detailed description, and from the claims.

### DETAILED DESCRIPTION

We have discovered that the combination of kinesin inhibitor and antiproliferative agent of Group A results in improved antiproliferative activity against neoplasms over the administration of either agent alone. Phenothiazine class kinesin inhibitors, such as chlorpromazine, are used as antipsychotic agents. Phenothiazines are thought to elicit their antipsychotic and antiemetic effects via interference with central dopaminergic pathways in the mesolimbic and medullary chemoreceptor trigger zone areas of the brain, respectively. Structural and functional analogs of chlorpromazine are known and, based on known properties that are shared between chlorpromazine and its analogs and metabolites, any of these analogs or metabolites can be substituted for chlorpromazine in the antiproliferative combinations of the invention.

Phenothiazines

Phenothiazines that are useful in the antiproliferative combination of the invention are without limitation, the compounds described in structure (I) above, such as acepromazine, cyamemazine, fluphenazine, mepazine, methotrimeprazine, methoxypromazine, perazine, pericyazine, perimethazine, perphenazine, pipamazine, pipazethate, piperacetazine, pipotiazine, prochlorperazine, promethazine, propionylpromazine, propiomazine, sulforidazine, thiazinaminiumsalt, thiethylperazine, thiopropazate, thioridazine, trifluoperazine, trimeprazine, thioproperazine, trifluomeprazine, triflupromazine, chlorpromazine, chlorproethazine, those compounds in PCT publication WO02/057244, and those compounds in U.S. Pat. No. 2,415,363; U.S. Pat. No. 2,519,886; U.S. Pat. No. 2,530,451; U.S. Pat. No. 2,607,773; U.S. Pat. No. 2,645,640; U.S. Pat. No. 2,766,235; U.S. Pat. No. 2,769,002; U.S. Pat. No. 2,784,185; U.S. Pat. No. 2,785,160; U.S. Pat. No. 2,837,518; U.S. Pat. No. 2,860,138; U.S. Pat. No. 2,877,224; U.S. Pat. No. 2,921,069; U.S. Pat. No. 2,957,870; U.S. Pat. No. 2,989,529; U.S. Pat. No. 3,058,979; U.S. Pat. No. 3,075,976; U.S. Pat. No. 3,194,733; U.S. Pat. No. 3,350,268; U.S. Pat. No. 3,875,156; U.S. Pat. No. 3,879,551; U.S. Pat. No. 3,959,268; U.S. Pat. No. 3,966,930; U.S. Pat. No. 3,998,820; U.S. Pat. No. 4,785,095; U.S. Pat. No. 4,514,395; U.S. Pat. No. 4,985,559; U.S. Pat. No. 5,034,019; U.S. Pat. No. 5,157,118; U.S. Pat. No. 5,178,784; U.S. Pat. No. 5,550,143; U.S. Pat. No. 5,595,989; U.S. Pat. No. 5,654,323; U.S. Pat. No. 5,688,788; U.S. Pat. No. 5,693,649; U.S. Pat. No. 5,712,292; U.S. Pat. No. 5,721,254; U.S. Pat. No. 5,795,888; U.S. Pat. No. 5,597,819; U.S. Pat. No. 6,043,239; and U.S. Pat. No. 6,569,849, each of which is incorporated herein by reference. Structurally related phenothiazines having similar antiproliferative properties are also intended to be encompassed by this group.

Phenothiazine compounds are described by formula (I), above.

The most commonly prescribed member of the phenothiazine family is chlorpromazine, which has the structure:

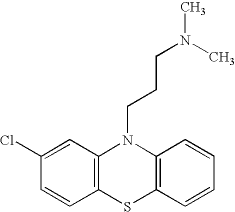

Chlorpromazine is currently available in the following forms: tablets, capsules, suppositories, oral concentrates and syrups, and formulations for injection.

Phenothiazines considered to be chlorpromazine analogs include fluphenazine, prochlorperazine, promethazine, thioridazine, and trifluoperazine. Many of these share antipsychotic or antiemetic activity with chlorpromazine. Also included as chlorpromazine analogs are those compounds in PCT Publication No. WO02/057244, which is hereby incorporated by reference.

Phenothiazines are also known to inhibit the activity of protein kinase C. Protein kinase C mediates the effects of a large number of hormones and is involved in may aspects of cellular regulation and carcinogenesis (Castagna, et al., J. Biol. Chem. 1982, 257:7847-51). The enzyme is also thought to play a role in certain types of resistance to cancer chemotherapeutic agents. Chlorpromazine has been investigated for the inhibition of protein kinase C both in vitro (Aftab, et al., Mol. Pharmacology, 1991, 40:798-805) and in vivo (Dwivedi, et al., J. Pharm. Exp. Ther., 1999, 291:688-704). Phenothiazines are also known as calmodulin inhibitors and mitotic kinesin inhibitors, the better of which modulate the movements of spindles and chromosomes in dividing cells.

Chlorpromazine also has strong alpha-adrenergic blocking activity and can cause orthostatic hypotension. Chlorpromazine also has moderate anticholinergic activity manifested as occasional dry mouth, blurred vision, urinary retention, and constipation. Chlorpromazine increases prolactin secretion owing to its dopamine receptor blocking action in the pituitary and hypothalamus.

Chlorpromazine is readily absorbed from the gastrointestinal tract. Its bioavailability is variable due to considerable first pass metabolism by the liver. Liquid concentrates may have greater bioavailability than tablets. Food does not appear to affect bioavailability consistently. I.m. administration bypasses much of the first pass effect and higher plasma concentrations are achieved. The onset of action after i.m. administration is usually 15 to 30 minutes and after oral administration 30 to 60 minutes. Rectally administered chlorpromazine usually takes longer to act than orally administered chlorpromazine.

Chlorpromazine Metabolites

Because chlorpromazine undergoes extensive metabolic transformation into a number of metabolites that may be therapeutically active, these metabolites may be substituted from chlorpromazine in the antiproliferative combination of the invention. The metabolism of chlorpromazine yields, for example, oxidative N-demethylation to yield the corresponding primary and secondary amine, aromatic oxidation to yield a phenol, N-oxidation to yield the N-oxide, S-oxidation to yield the sulphoxide or sulphone, oxidative deamination of the aminopropyl side chain to yield the phenothiazine nuclei, and glucuronidation of the phenolic hydroxy groups and tertiary amino group to yield a quaternary ammonium glucuronide.

In other examples of chlorpromazine metabolites useful in the antiproliferative combination of the invention, each of positions 3, 7, and 8 of the phenothiazine can independently be substituted with a hydroxyl or methoxyl moiety.

Therapy

The combinations of the invention are useful for the treatment of neoplasms. Therapy may be performed alone or in conjunction with another therapy (e.g., surgery, radiation therapy, immunotherapy, or gene therapy). Additionally, a person having a greater risk of developing a neoplasm (e.g., one who is genetically predisposed or one who previously had a neoplasm) may receive prophylactic treatment to inhibit or delay neoplastic formation. The duration of the combination therapy depends on the type of disease or disorder being treated, the age and condition of the patient, the stage and type of the patient's disease, and how the patient responds to the treatment. Therapy may be given in on-and-off cycles that include rest periods so that the patient's body has a chance to recovery from any as yet unforeseen side-effects.

Examples of cancers and other neoplasms that can be treated using the methods and compositions of the invention include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, and retinoblastoma).

Combination therapy may be provided wherever chemotherapy is performed: at home, the doctor's office, a clinic, a hospital's outpatient department, or a hospital. Treatment generally begins at a hospital so that the doctor can observe the therapy's effects closely and make any adjustments that are needed. The duration of the combination therapy depends on the kind of neoplasm being treated, the age and condition of the patient, the stage and type of the patient's disease, and how the patient's body responds to the treatment. Drug administration may be performed at different intervals (e.g., daily, weekly, or monthly) and the administration of each agent can be determined individually. Combination therapy may be given in on-and-off cycles that include rest periods so that the patient's body has a chance to build healthy new cells and regain its strength.

Depending on the type of cancer and its stage of development, the combination therapy can be used to treat cancer, to slow the spreading of the cancer, to slow the cancer's growth, to kill or arrest cancer cells that may have spread to other parts of the body from the original tumor, to relieve symptoms caused by the cancer, or to prevent cancer in the first place. Combination therapy can also help people live more comfortably by eliminating cancer cells that cause pain or discomfort.

For example, a patient suffering from a non small cell lung carcinoma or colon adenocarcinoma, can be treated by a combination of chlorpromazine and a Group A antiproliferative. The selection of an antiproliferative agent can be determined by the attending physician. Accordingly, the physician may determine that an antimitotics agent is required and, by referring to the drugs listed in Group A, select any one of the agents listed under the heading antimitotic agents, or more specifically microtubule binding, stabilizing agent (e.g., docetaxel). The physician can further select the specific drug depending on the stage or type of cancer being treated. For metastatic cancers, the choice of the drug to be used in combination with chlorpromazine may be metalloproteinase inhibitors (e.g., neovastat). The physician, at his/her discretion can further refer to the Table 1 and further prescribe an additional drug. The additional drug can be another antimitotic agent (e.g., vinblastine), or a drug selected from a mechanistic pathway inhibitor (e.g., tyrosine kinase inhibitor, such as, tyrphostin) that will aid in halting the proliferation of the neoplasm or maximally alleviate the suffering of the patient.

The administration of a combination of the present invention allows for the administration of lower doses of each compound, providing similar efficacy and lower toxicity compared to administration of either compound alone. Alternatively, such combinations result in improved efficacy in treating neoplasm with similar or reduced toxicity over the single agent alone, at moderate or high doses.

Formulation of Pharmaceutical Compositions

The administration of each compound of the combination may be by any suitable means that results in a concentration of the compound that, combined with the other component, is anti-neoplastic upon reaching the target region. The compound may be contained in any appropriate amount in any suitable carrier substance, and is generally present in an amount of 1-95% by weight of the total weight of the composition. The composition may be provided in a dosage form that is suitable for the oral, parenteral (e.g., intravenously, intramuscularly), rectal, cutaneous, nasal, vaginal, inhalant, skin (patch), or ocular administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The pharmaceutical compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

Each compound of the combination may be formulated in a variety of ways that are known in the art. For example, the first and second agents may be formulated together or separately. Desirably, the first and second agents are formulated together for the simultaneous or near simultaneous administration of the agents. Such co-formulated compositions can include the phenothiazine and antiproliferative agent formulated together in the same pill, capsule, liquid, etc.

Administration of each compound in controlled release formulations is useful where the compound of formula (I) or the antiproliferative agent, has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD₅₀) to median effective dose (ED₅₀)); (ii) a narrow absorption window in the gastro-intestinal tract; (iii) a short biological half-life; or (iv) the pharmacokinetic profile of each component must be modified to maximize the exposure of the neoplasm to an amount of each agent, together, that is therapeutically effective. Accordingly, a sustained release formulation may be used to avoid frequent dosing that may be required in order to sustain the plasma levels of both agents at a therapeutic level.

Many strategies can be pursued to obtain controlled release in which the rate of release outweighs the rate of metabolism of the therapeutic compound. For example, controlled release can be obtained by the appropriate selection of formulation parameters and ingredients (e.g., appropriate controlled release compositions and coatings). Examples include single or multiple unit tablet or capsule compositions, oil solutions, suspensions, emulsions, microcapsules, microspheres, nanoparticles, patches, and liposomes. The control release mechanism can be such that the compound of formula (I) is released first, followed by the Group A antiproliferative agent. The release mechanism can also be controlled that the two drugs are released at period intervals, the release could be simultaneous or a delayed release of one, when release of a particular drug is preferred over the other.

Controlled release formulations may include a degradable or nondegradable polymer, hydrogel, organogel, or other physical construct that modifies the bioabsorption, half life or biodegradation of the agent. The controlled release formulation can be a material that is painted or otherwise applied onto the afflicted site, either internally or externally. In one example, the invention provides a biodegradable bolus or implant that is inserted into the pocket created by surgical resection of a tumor, or directly into the tumor itself. In another example, the controlled release formulation can be applied to a basal cell carcinoma or squamous cell carcinoma. In yet another example, hydrogels, such as those described in U.S. Pat. No. 5,626,863 can be used in controlled release formulations of compostions of the invention.

These polymers can be tailored to degrade at a desired rate and with a desired kinetics by selecting the appropriate monomers, method of preparation and molecular weight. Differences in crystallinity of the monomer can alter the polymeric degradation rate. Due to the relatively hydrophobic nature of most polymers, actual mass loss can begin with the oligomeric fragments that are small enough to be water soluble; hence, even the initial molecular weight can influence the degradation rate.

The individually or separately formulated agents can be packaged together as in a kit. Non limiting examples include kits that contain, e.g., two pills, a pill and a powder, a suppository and a liquid in a vial, two topical creams, among others. The kit can include optional components that aid in the administration of the unit dose to patients, such as vials for reconstituting powder forms, syringes for injection, customized IV delivery systems, inhalers, among others. Additionally, the unit dose kit can contain instructions for preparation and administration of the compositions. The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

Dosages

The dosage of each compound or agent of the claimed combinations depends on several factors, including: the administration method, the neoplasm to be treated, the severity of the neoplasm, whether the neoplasm is to be treated or prevented, and the race, gender, age, weight, site of the neoplasm, and health of the patient to be treated.

An antiproliferative agent of the invention is usually given by the same route of administration that is known to be effective for delivering it as a monotherapy. When used in combination therapy with a kinesin inhibitor or compound of formula (I) according to the methods of this invention, the antiproliferative agent can be administered at the dosage recommended by the Physician's Desk Reference, 57^{th} Edition (2003) or in amounts and frequencies less than those recommended for monotherapeutic use.

Chlorpromazine Dosage

For chlorpromazine, oral dosage is normally about 0.1 mg to 300 mg per dose administered (preferably about 1 mg to 100 mg) one to four times daily for one day to one year and may be administered for the life of the patient. Administration may also be given in cycles, such that there are periods during which time chlorpromazine is not administered. This period could be, for example, about a day, a week, a month, or a year or more.

The rectal dosage of chlorpromazine is as described for orally administered chlorpromazine.

For intravenous or intramuscular administration of chlorpromazine, a daily dose of about 0.05 mg/kg to about 20 mg/kg is recommended, a dose of about 0.05 mg/kg to about 10 mg/kg is preferred, and a dose of about 0.1 mg/kg to about 4 mg/kg is most preferred. Intravenous or intramuscular administration is usually daily for up to about 6 to 12 months or more. It may be desirable to administer a compound over a one to three hour period; this period may be extended to last 24 hours or more. As is described for oral administration, there may be periods of about one day to one year or longer during which at least one of the drugs is not administered.

For inhalation, chlorpromazine is administered at a dose of about 1 mg to 1000 mg, and preferably at a dose of 2 mg to 600 mg, is administered daily.

For topical administration of chlorpromazine, a dose of about 1 mg to about 5 g administered one to ten times daily for one week to 12 months is usually preferable.

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

### EXAMPLES

### Tumor Cell Culture

Human non-small lung carcinoma cells A549 (ATCC# CCL-185) or HCT116, (ATCC #CCL-247) colon adenocarcinoma cells, were grown at 37±0.5° C. and 5% CO₂ in RPMI 1640 supplemented with 10% FBS, 2 mM glutamine, 1 % penicillin, and 1% streptomycin.

### Test Compounds

Chlorpromazine, dacarbazine, vinblastine, hydroxycamptothecin, leucovorin, and paclitaxel were obtained from Sigma Chemical Co. (St. Louis, Mo.). Docetaxel and vinorelbine were obtained from Sequoia, Research Products, Inc., Oxford UK. Floxuridine was obtained from MicroSource Discovery Systems, Inc. Gaylordsville, Conn. Tyrphostin AG1478 was obtained from Biomol, Plymouth Meeting, Pa.

Stock solutions (1000×) of each compound were prepared in DMSO and stored at −20° C. Master stock plates of 2-fold or 4-fold serial dilutions of individual compounds were prepared in 384-well plates. Combination matrices of test compounds were generated from these master stock plates by dilution into growth media described above. The final concentration of test compounds in the combination matrices was 10× greater than used in the assay. The combination matrices were used immediately and discarded.

### Anti-proliferation Assay

The anti-proliferation assays were performed in 384-well plates. The tumor cells were liberated from the culture flask using a solution of 0.25% trypsin. Cells were diluted in culture media such that 1500 cells were delivered in 40 μL of media into each assay well. Asay plates were incubated 16-24 hours at 37±0.5 C with 5% CO₂. 10× stock solutions (4.5 μL) from the combination matrices were added to 40 μL of culture media in assay wells. Assay plates were incubated for 72-80 hours at 37±0.5° C. with 5% CO₂. Forty microliters of 105% Alamar Blue in growth media warmed to 37±0.5° C. was added to each assay well following the incubation period. Alamar Blue metabolism was quantified by the amount of fluorescence intensity 3.5-5.0 hours after addition. Quantification, using an LJL Analyst AD reader (LJL Biosystems), was taken in the middle of the well with high attenuation, a 100 msec read time, an excitation filter at 530 nm, and an emission filter at 575 nm. For some experiments, quantification was performed using a Wallac Victor² reader. Measurements were taken at the top of the well with stabilized energy lamp control; a 100 msec read time, an excitation filter at 530 nm, and an emission filter at 590 nm. No significant differences between plate readers were measured.

The percent inhibition (%I) for each well was calculated using the following formula:
%I=[(avg. untreated wells−treated well)/(avg. untreated wells)]×100

The average untreated well value (avg. untreated wells) is the arithmetic mean of 32-90, wells depending on plate format, from the same assay plate treated with vehicle alone. Negative inhibition values result from local variations in treated wells as compared to untreated wells.

### Example 1

### Antiproliferative Activity of Chlorpromazine and Dacarbazine Against HCT116 Colon Adenocarcinoma Cells

Inhibition of proliferation was measured by anti-proliferation assay as described below after incubation with the test compound(s) for 72 hours. The effects of varying concentrations of chlorpromazine, dacarbazine, or a combination of chlorpromazine and dacarbazine were compared to control wells (seeded with HCT116 cells, but incubated with vehicle).

The results of this experiment are shown in Table 2. The effects of the agents alone and in combination are shown as percent inhibition of cell proliferation.

TABLE 2
Percent inhibition of Alamar Blue Metabolism in HCT116 cells

| | |
|---|---|
| Chlorpro- | |
| mazine | Dacarbazine (μM) |

| (μM) | 0 | 3.4 | 6.9 | 14 | 27 | 55 |
|---|---|---|---|---|---|---|
| 0 | −7.74 | 41.5 | 26.1 | 42.6 | 64.6 | 74.2 |
| 2.5 | 4.03 | 59.3 | 60 | 48.1 | 64.3 | 76.5 |
| 5 | 17 | 55.9 | 32.9 | 61.1 | 63.9 | 72 |
| 10 | 54.6 | 71.5 | 71.3 | 74.9 | 80.9 | 80.6 |
| 20 | 91.2 | 88.4 | 88.5 | 86 | 91.3 | 92.3 |
| 40 | 95.8 | 96.1 | 96.1 | 96.2 | 96.5 | 95.9 |

### Example 2

### Antiproliferative Activity of Chlorpromazine and Floxuridine Against HCT116 Cells

Table 3 shows the results from an anti-proliferation assay using HCT116 cells treated with chlorpromazine, floxuridine, or a combination of chlorpromazine and floxuridine. The effects of the agents alone and in combination are shown as percent inhibition of cell proliferation.

TABLE 3
Percent inhibition of Alamar Blue Metabolism in HCT116 cells

| Chlorpro- | | | | | | |
|---|---|---|---|---|---|---|
| mazine | Floxuridine (μM) | | | | | |
| (μM) | 0 | 0.32 | 0.63 | 1.3 | 2.5 | 5.1 |
| 0 | −2.69 | 78.9 | 80.6 | 80.1 | 83.7 | 87.1 |
| 2.5 | −0.0804 | 71.8 | 75.5 | 80.8 | 84 | 86.4 |
| 5 | 3.53 | 64 | 78.4 | 83.2 | 90.9 | 87.6 |
| 10 | 65.4 | 84.8 | 78.2 | 85.4 | 86.9 | 84.6 |
| 20 | 88.5 | 89 | 87.1 | 88.4 | 91.7 | 91.4 |
| 40 | 95.7 | 95.5 | 95.7 | 95.7 | 95.7 | 95.7 |

### Example 3

### Antiproliferative Activity of Chlorpromazine and Tyrphostin 1486 Against Human HCT116 Cells

Table 4 shows the results from an anti-proliferation assay using HCT116 cells treated with chlorpromazine, tyrphostin, or a combination of chlorpromazine and tyrphostin. The effects of the agents alone and in combination are shown as percent inhibition of cell proliferation.

TABLE 4
Percent inhibition of Alamar Blue Metabolism in HCT116 cells

| | |
|---|---|
| Chlorpro- | |
| mazine | Tyrphostin 1486 (μM) |

| (μM) | 0 | 3.2 | 6.4 | 13 | 26 | 51 |
|---|---|---|---|---|---|---|
| 0 | 36.3 | 5.45 | 42.7 | 78.7 | 81.4 | 81.3 |
| 7 | −18.6 | 2.96 | 54.4 | 77 | 92.9 | 80.4 |
| 14 | −12.5 | 32.6 | 74.7 | 81.2 | −6.01 | 82.5 |
| 28 | 62.2 | 76.7 | 83 | 83.3 | 83.6 | 83 |
| 56 | 85.6 | 87.1 | 87.3 | 87.2 | 87 | 86.6 |
| 113 | 95.9 | 96 | 95.6 | 95.4 | 94.6 | 94 |

### Example 4

### Antiproliferative Activity of Chlorpromazine and Vinblastine Against HCT116 Cells

Table 5 shows the results from an anti-proliferation assay using HCT116 cells treated with chlorpromazine, vinblastine, or a combination of chlorpromazine and vinblastine. The effects of the agents alone and in combination are shown as percent inhibition of cell proliferation.

TABLE 5
Percent inhibition of Alamar Blue Metabolism in HCT116 cells

| Chlorpro- | | | | | | |
|---|---|---|---|---|---|---|
| mazine | Vinblastine (μM) | | | | | |
| (μM) | 0 | 0.00068 | 0.0014 | 0.0027 | 0.0054 | 0.011 |
| 0 | −20 | −5.37 | −7.03 | −20 | 41.3 | 68.2 |
| 2.5 | −20 | −20 | −20 | 5.33 | 67.8 | 72.1 |
| 5 | −20 | −20 | 14.4 | 66.3 | 72.1 | 75.4 |
| 10 | 50.8 | 72.5 | 81.2 | 81.3 | 82 | 80.2 |
| 20 | 85.8 | 89 | 87.6 | 90.2 | 83.7 | 85.2 |
| 40 | 94.5 | 95.4 | 94.9 | 95 | 95 | 95 |

### Example 5

### Antiproliferative Activity of Chlorpromazine and Vinorelbine Against HCT116 Cells

Table 6 shows the results from an anti-proliferation assay using HCT116 cells treated with chlorpromazine, vinorelbine, or a combination of chlorpromazine and vinorelbine. The effects of the agents alone and in combination are shown as percent inhibition of cell proliferation.

TABLE 6
Percent inhibition of Alamar Blue Metabolism in HCT116 cells

| Chlorpro- | | | | | | |
|---|---|---|---|---|---|---|
| mazine | Vinorelbine (μM) | | | | | |
| (μM) | 0 | 0.004 | 0.008 | 0.016 | 0.032 | 0.064 |
| 0 | 3.21 | −8.91 | 42.1 | 66 | 64 | 68.8 |
| 2.5 | −0.591 | 30.2 | 65.2 | 62.3 | 70 | 74 |
| 5 | −20 | 66.9 | 72.3 | 75.5 | 74.7 | 69 |
| 10 | 60 | 86.9 | 84.1 | 82.5 | 79.5 | 69 |
| 20 | 88.4 | 88.3 | 85.1 | 87.5 | 87.9 | 82.6 |
| 40 | 94.7 | 94.9 | 94.8 | 94.9 | 94.8 | 94.9 |

### Example 6

### Antiproliferative Activity of Chlorprmazine and Hydroxycamptothecin Against HCT116 Cells

The results from a chlorprmazine and hydroxycamptothecin combination on HCT116 cell growth are shown in Table 7. The effects of the agents alone and in combination are shown as percent inhibition of cell proliferation.

TABLE 7
Percent inhibition of Alamar Blue Metabolism in HCT116 cells

| Chlorpro- | | | | | | |
|---|---|---|---|---|---|---|
| mazine | Hydroxycamptothecin (μM) | | | | | |
| (μM) | 0 | 0.015 | 0.029 | 0.059 | 0.12 | 0.24 |
| 0 | −17.3 | 84.9 | 88.3 | 91 | 93 | 88.5 |
| 2.5 | −8.14 | 82.4 | 88.4 | 91.1 | 93.2 | 90.9 |
| 5 | 24 | 81 | 88.1 | 90.4 | 92.9 | 91.5 |
| 10 | 84 | 81.5 | 86.1 | 92.8 | 93.1 | 90.4 |
| 20 | 88.2 | 93.3 | 93.7 | 94.8 | 95 | 93.6 |
| 40 | 95.8 | 95.8 | 95.6 | 95.7 | 95.7 | 95.6 |

### Example 7

### Antiproliferative Activity of Chlorpromazine and Leucovorin Against HCT116 Cells

The results from a chlorpromazine and leucovorin combination on HCT116 cell growth are shown in Table 8. The effects of the agents alone and in combination are shown as percent inhibition of cell proliferation.

TABLE 8
Percent inhibition of Alamar Blue Metabolism in HCT116 cells

| Chlorpro- | | | | | | |
|---|---|---|---|---|---|---|
| mazine | Leucovorin (μM) | | | | | |
| (μM) | 0 | 1.2 | 2.4 | 4.9 | 9.8 | 20 |
| 0 | −17.5 | −16.9 | −2.21 | −12.2 | −9.22 | −11.5 |
| 2.5 | −0.0253 | −11.7 | −14.7 | −11.2 | −8.3 | −3.89 |
| 5 | −4.39 | 0.733 | −9.82 | 37.4 | 2.29 | 6.63 |
| 10 | 3.27 | 45.8 | 37.1 | 50.8 | 42.2 | 70.5 |
| 20 | 80.9 | 86.3 | 83.4 | 90.6 | 91.3 | 90.4 |
| 40 | 95.6 | 95.9 | 96 | 95.2 | 96 | 96 |

### Example 8

### Antiproliferative Activity of Chlorpromazine and Paclitaxel Against GCT116 Cells

Table 9 shows the results from an anti-proliferation assay using HCT116 cells treated with chlorpromazine, paclitaxel, or a combination of chlorpromazine and paclitaxel. The effects of the agents alone and in combination are shown as percent inhibition of cell proliferation.

TABLE 9
Percent inhibition of Alamar Blue Metabolism in HCT116 cells

| Chlorpro- | | | | | | |
|---|---|---|---|---|---|---|
| mazine | Paclitaxel (μM) | | | | | |
| (μM) | 0 | 0.0018 | 0.0037 | 0.0073 | 0.015 | 0.029 |
| 0 | 34.6 | 46.1 | 70.4 | 78.8 | 84.6 | 88.8 |
| 2.5 | 10.1 | 65.4 | 70.7 | 80.7 | 85.5 | 86.1 |
| 5 | −11.8 | 26.4 | 72.7 | 81.6 | 88.9 | 87.4 |
| 10 | 56.2 | 32.9 | 80.6 | 84.9 | 89.2 | 85.7 |
| 20 | 87 | 80.4 | 88.8 | 87.9 | 89.3 | 90.1 |
| 40 | 95.7 | 95.9 | 95.8 | 95.8 | 95.9 | 95.8 |

### Example 9

### Antiproliferative Activity of Chlorpromazine and Docetaxel Against HCT116 Cells

Table 10 shows the results from an anti-proliferation assay using HCT116 cells treated with chlorpromazine, docetaxel, or a combination of chlorpromazine, and docetaxel. The effects of the agents alone and in combination are shown as percent inhibition of cell proliferation.

TABLE 10
Percent inhibition of Alamar Blue Metabolism in HCT116 cells

| Chlorpro- | | | | | | |
|---|---|---|---|---|---|---|
| mazine | Docetaxel (μM) | | | | | |
| (μM) | 0 | 0.00076 | 0.0015 | 0.0031 | 0.0061 | 0.012 |
| 0 | −1.47 | 47.4 | 63.6 | 73.6 | 77.3 | 80.6 |
| 2.5 | −12.2 | 42.8 | 70 | 73.5 | 75.2 | 80.9 |
| 5 | 2.8 | 58.6 | 74.8 | 77.5 | 80.5 | 82.3 |
| 10 | 84.8 | 80.8 | 73.9 | 83.8 | 85.5 | 79.7 |
| 20 | 91.9 | 90 | 89.5 | 94.9 | 87 | 90.1 |
| 40 | 95.7 | 95.9 | 95.7 | 95.9 | 96 | 95.9 |

Example 10

### Antiproliferative Activity in Other Cell Lines

The anti-proliferative effect demonstrated with the tumor cell lines used herein can be similarly demonstrated using other cancer cell lines, such as NSC lung carcinoma, MCF7 mammary adenocarcinoma, PA-1 ovarian teratocarcinoma, HT29 colorectal adenocarcinoma, H1299 large cell carcinoma, U-2 OS osteogenic sarcoma, U-373 MG glioblastoma, Hep-3B hepatocellular carcinoma, BT-549 mammary carcinoma, T-24 bladder cancer, C-33A cervical carcinoma, HT-3 metastatic cervical carcinoma, SiHa squamous cervical carcinoma, CaSki epidermoid cervical carcinoma, NCI-H292 mucoepidermoid lung carcinoma, NCI-2030, non small cell lung carcinoma, HeLa, epithelial cervical adenocarcinoma, KB epithelial mouth carcinoma, HT1080 epithelial fibrosarcoma, Saos-2 epithelial osteogenic sarcoma, PC3 epithelial prostate adenocarcinoma, SW480 colorectal carcinoma, CCL-228, MS-751 epidermoid cervical carcinoma, LOX IMVI melanoma, MALME-3M melanoma, M14 melanoma, SK-MEL-2 melanoma, SK-MEL-28 melanoma, SK-MEL-5 melanoma, UACC-257 melanoma, and UACC-62 melanoma cell lines. The specificity can be tested by using cells such as NHLF lung fibroblasts, NHDF dermal fibroblasts, HMEC mammary epithelial cells, PrEC prostate epithelial cells, HRE renal epithelial cells, NHBE bronchial epithelial cells, CoSmC Colon smooth muscle cells, CoEC colon endothelial cells, NHEK epidermal keratinocytes, and bone marrow cells as control cells.

### OTHER EMBODIMENTS

All publications and patents cited in this specification are incorporated herein by reference as if each individual publication or patent were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method for treating a patient diagnosed with or at risk of developing a neoplasm, said method comprising administering to said patient:
(a) a compound having the formula (I): 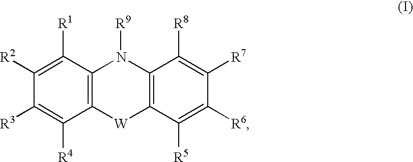
or a pharmaceutically acceptable salt thereof,
wherein R² is CF₃, halogen, OCH₃, COCH₃, CN, OCF₃, COCH₂CH₃, CO(CH₂)₂CH₃, or SCH₂CH₃;
R⁹ is selected from: 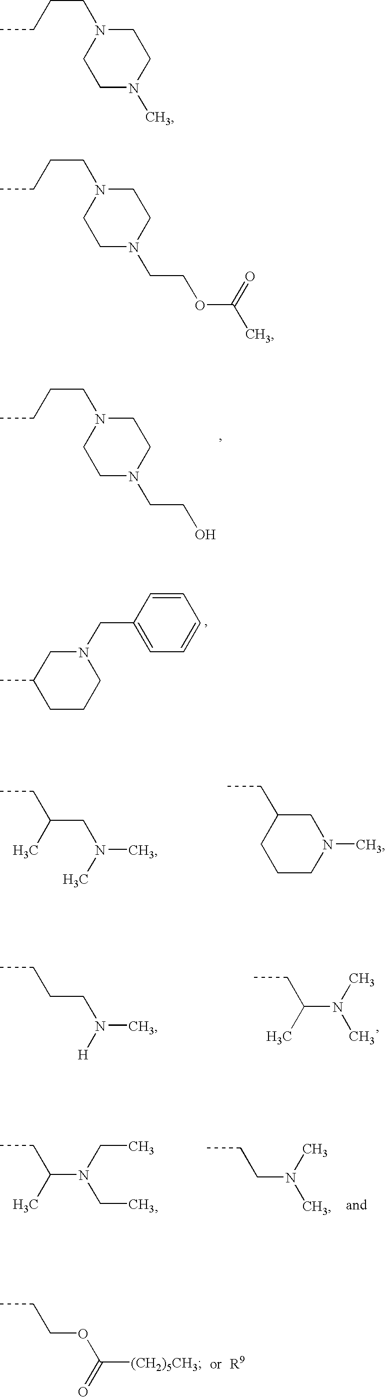
has the formula: 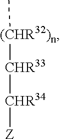
wherein n is 0 or 1, Z is NR³⁵R³⁶ or OR³⁷; each of R³², R³³, R³⁴, R³⁵, R³⁶, and R³⁷ is, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, acyl, or C₁₋₇ heteroalkyl; or any of R³³, R³⁴, R³⁵, R³⁶, and R³⁷ can be optionally taken together with intervening carbon or non-vicinal O, S, or N atoms to form one or more five- to seven-membered rings, optionally substituted by H, halogen, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, acyl, or C₁₋₇ heteroalkyl;
each of R¹, R³, R⁴, R⁵, R⁶, R⁷, and R⁸ is independently H, OH, F, OCF₃, or OCH₃; and
W is NO, 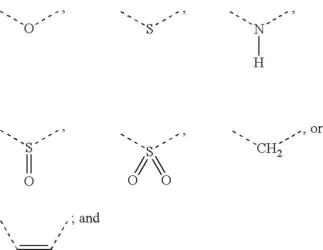
(b) a Group A antiproliferative agent,
wherein said compound of formula (I) and said Group A antiproliferative agent are administered simultaneously, or within 14 days of each other, in amounts that together are sufficient to inhibit the growth of said neoplasm, and with the proviso that said method does not include administering a bis-benzimidazole compound, an endo-exonuclease inhibitor, a PRL phosphatase inhibitor, or a PTP1B inhibitor within 20 days of administering said compound of formula (I).

2. The method of claim 1, wherein when the compound of formula (I) is trifluoperazine, the antiproliferative agent is not doxorubicin, aclacinomycin, trifluoroacetyladriamycin-14-valerate, vinblastine, dactinomycin, colchicine, or adriamycin, and when the compound of formula (I) is chlorpromazine, the antiproliferative agent is not paclitaxel, doxorubicin, vinblastine, dactinomycin, or colchicine, and when the compound of formula (I) is thioridazine, the antiproliferative agent is not doxorubicin, vinblastine, dactinomycin, or colchicine.

3. The method of claim 1, wherein said Group A antiproliferative agent is dacarbazine, mitoxantrone, bicalutamide, floxuridine, leucovorin, vinblastine, vinorelbine, hydroxycamptothecin, tyrphostin, docetaxel, or combinations thereof.

4. The method of claim 1, wherein the antiproliferative agent is carmustine, cisplatin, etoposide, melphalan, mercaptopurine, methotrexate, mitomycin, vinblastine, paclitaxel, docetaxel, vincristine, vinorelbine, cyclophosphamide, chlorambucil, gemcitabine, capecitabine, 5-fluorouracil, fludarabine, raltitrexed, irinotecan, topotecan, doxorubicin, epirubicin, letrozole, anastrazole, formestane, exemestane, tamoxifen, toremofine, goserelin, leuporelin, bicalutamide, flutamide, nilutamide, hypericin, trastuzumab, rituximab, or combinations thereof.

5. The method of claim 1, wherein said compound of formula (I) is acepromazine, chlorfenethazine, chlorpromazine, N-methyl chlorpromazine, cyamemazine, fluphenazine, mepazine, methotrimeprazine, methoxypromazine, norchlorpromazine, perazine, perphenazine, phenothiazine, prochlorperazine, promethazine, propiomazine, putaperazine, thiethylperazine, thiopropazate, thioridazine, trifluoperazine, or triflupromazine.

6. The method of claim 1, wherein said compound of formula (I) and said antiproliferative agent are administered within ten days of each other.

7. The method of claim 6, wherein said compound of formula (I) and said antiproliferative agent are administered within five days of each other.

8. The method of claim 7, wherein said compound of formula (I) and said antiproliferative agent are administered within twenty-four hours of each other.

9. The method of claim 8, wherein said compound of formula (I) and said antiproliferative agent are administered simultaneously.

10. A method for treating a patient diagnosed with or at risk of developing a neoplasm, said method comprising administering to said patient:
(a) a kinesin inhibitor, and
(b) a Group A antiproliferative agent,
wherein said kinesin inhibitor and said Group A antiproliferative agent are administered simultaneously, or within 14 days of each other, in amounts that together are sufficient to inhibit the growth of said neoplasm and with the proviso that said method does not include administering a bis-benzimidazole compound, an endo-exonuclease inhibitor, a PRL phosphatase inhibitor, or a PTP1B inhibitor within 20 days of administering said kinesin inhibitor.

11. The method of claim 10, wherein when the kinesin inhibitor is trifluoperazine, the antiproliferative agent is not doxorubicin, aclacinomycin, trifluoroacetyladriamycin-14-valerate, vinblastine, dactinomycin, colchicine, or adriamycin, and when the kinesin inhibitor is chlorpromazine, the antiproliferative agent is not paclitaxel, doxorubicin, vinblastine, dactinomycin, or colchicine, and when the kinesin inhibitor is thioridazine, the antiproliferative agent is not doxorubicin, vinblastine, dactinomycin, or colchicine.

12. The method of claim 10, wherein said Group A antiproliferative agent is dacarbazine, mitoxantrone, bicalutamide, floxuridine, leucovorin, vinblastine, vinorelbine, hydroxycamptothecin, tyrphostin, docetaxel, or combinations thereof.

13. The method of claim 10, wherein said kinesin inhibitor is chlorpromazine or trifluoperazine.

14. The method of claims 1 or 10, wherein said neoplasm is cancer.

15. The method of claim 14, wherein said cancer is lung cancer.

16. The method of claim 14, wherein said cancer is colon cancer.

17. The method of claim 14, wherein said cancer is breast cancer.

18. The method of claim 14, wherein said cancer is prostate cancer.

19. The method of claim 14, wherein said cancer is acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia, polycythemia vera, Hodgkin's disease, non-Hodgkin's disease, Waldenstrom's macroglobulinemia, heavy chain disease, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, or retinoblastoma.

20. The method of claims 1 or 10, wherein said administering is intravenous, topical, subcutaneous, buccal, intramuscular, inhalation, rectal, or oral.

21. A composition comprising:
(a) a compound having the formula (I): 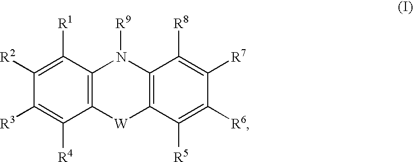
or a pharmaceutically acceptable salt thereof,
wherein R² is CF₃, halogen, OCH₃, COCH₃, CN, OCF₃, COCH₂CH₃, CO(CH₂)₂CH₃, or SCH₂CH₃;
R⁹ is selected from: 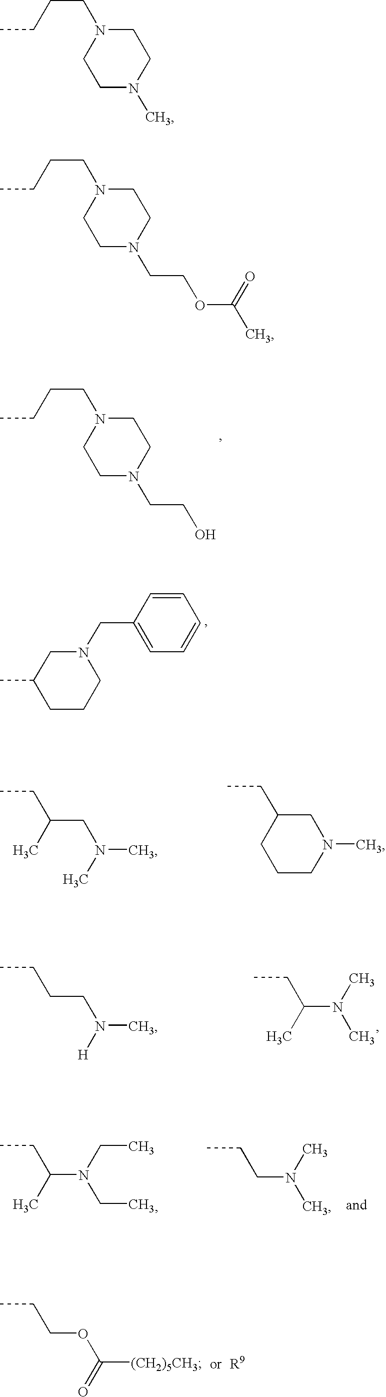
has the formula: 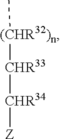
wherein n is 0 or , Z is NR³⁵ or OR³⁷; each of R³², R³³, R³⁴, R³⁵, R³⁶, and R³⁷ is, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, acyl, or C₁₋₇ heteroalkyl; or any of R³³, R^{34,} R³⁵, R³⁶ , and R³⁷ can be optionally taken together with intervening carbon or non-vicinal O, S, or N atoms to form one or more five- to seven-membered rings, optionally substituted by H, halogen, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, acyl, or C₁₋₇ heteroalkyl;
each of R¹, R³, R⁴, R⁵, R⁶, R⁷, and R⁸ is independently H, OH, F, OCF₃, or OCH₃; and
W is NO, 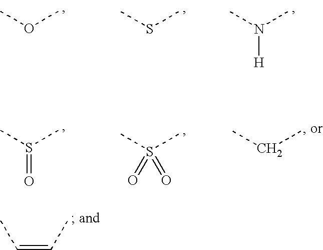
(b) a Group A antiproliferative agent,
wherein said compound of formula (I) and said Group A antiproliferative agent are present in amounts that together are sufficient to inhibit the growth of said neoplasm when administered to a patient, and with the proviso that said composition does not include a bis-benzimidazole compound, an endo-exonuclease inhibitor, a PRL phosphatase inhibitor, or a PTP1B inhibitor.

22. A composition consisting of one or more pharmaceutically acceptable excipients and a mixture of anti-neoplastic agents, wherein said mixture consists of:
(a) one or more compounds having the formula (I): 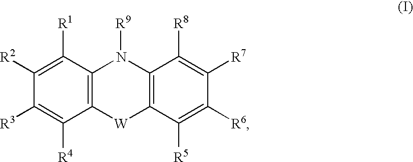
or a pharmaceutically acceptable salt thereof,
wherein R² is CF₃, halogen, OCH₃, COCH₃, CN, OCF₃, COCH₂CH₃, CO(CH₂)₂CH₃, or SCH₂CH₃;
R⁹ is selected from: 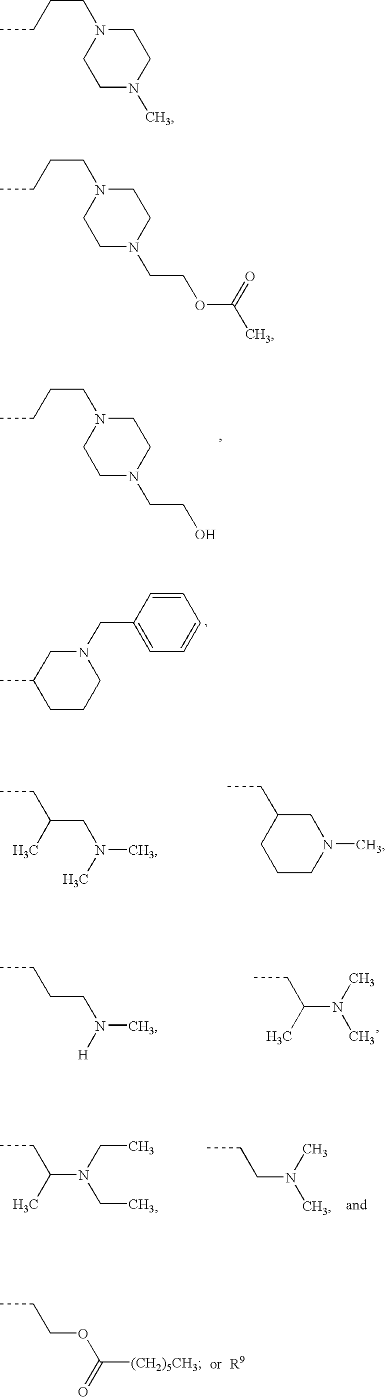
has the formula: 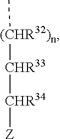
wherein n is 0 or 1, Z is NR³⁵R³⁶ or OR³⁷; each of R³², R³³, R³⁴, R³⁵, R³⁶, and R³⁷ is, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, acyl, or C₁₋₇ heteroalkyl; or any of R³³, R³⁴, R³⁵, R³⁶, and R³⁷ can be optionally taken together with intervening carbon or non-vicinal O, S, or N atoms to form one or more five- to seven-membered rings, optionally substituted by H, halogen, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, acyl, or C₁₋₇ heteroalkyl;
each of R¹, R³, R⁴, R⁵, R⁶, R⁷, and R⁸ is independently H, OH, F, OCF₃, or OCH₃; and
W is NO, 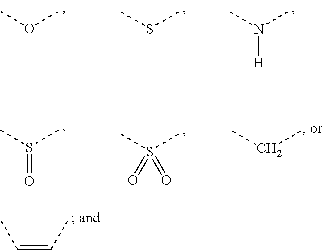
(b) one or more Group A antiproliferative agent(s), and
wherein said compound of formula (I) and said Group A antiproliferative agent are present in amounts that together are sufficient to inhibit the growth of said neoplasm when administered to a patient.

23. The composition of claims 21 or 22, wherein said Group A antiproliferative agent is dacarbazine, mitoxantrone, bicalutamide, floxuridine, vinorelbine, leucovorin, vinblastine hydroxycamptothecin, tyrphostin, or docetaxel.

24. The composition of claims 21 or 22, wherein the compound of formula (I) from is acepromazine, chlorpromazine, cyamemazine, fluphenazine, mepazine, methotrimeprazine, methoxypromazine, perazine, perphenazine, prochlorperazine, promethazine, propiomazine, thiethylperazine, thiopropazate, thioridazine, trifluoperazine, triflupromazine, or combinations thereof.

25. The composition of claims 21 or 22, wherein the antiproliferative agent is carmustine, cisplatin, etoposide, melphalan, mercaptopurine, methotrexate, mitomycin, vinblastine, paclitaxel, docetaxel, vincristine, vinorelbine, cyclophosphamide, chlorambucil, gemcitabine, capecitabine, 5-fluorouracil, fludarabine, raltitrexed, irinotecan, topotecan, doxorubicin, epirubicin, letrozole, anastrazole, formestane, exemestane, tamoxifen, toremofine, goserelin, leuporelin, bicalutamide, flutamide, nilutamide, hypericin, trastuzumab, rituximab, or combinations thereof.

26. A composition comprising:
(a) a kinesin inhibitor, and
(b) a Group A antiproliferative agent,
wherein said kinesin inhibitor and said Group A antiproliferative agent are present in amounts that together are sufficient to inhibit the growth of said neoplasm when administered to a patient, and with the proviso that said composition does not include a bis-benzimidazole compound, an endo-exonuclease inhibitor, a PRL phosphatase inhibitor, or a PTP1B inhibitor.

27. A composition consisting one or more pharmaceutically acceptable excipients and a mixture of anti-neoplastic agents, wherein said mixture consists of:
(a) a kinesin inhibitor, and
(b) a Group A antiproliferative agent,
wherein said kinesin inhibitor and said Group A antiproliferative agent are present in amounts that together are sufficient to inhibit the growth of said neoplasm when administered to a patient.

28. A kit comprising:
(a) a compound having the formula (I): 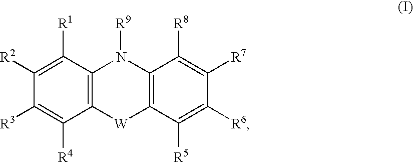
or a pharmaceutically acceptable salt thereof,
wherein R² is CF₃, halogen, OCH₃, COCH₃, CN, OCF₃, COCH₂CH₃, CO(CH₂)₂CH₃, or SCH₂CH₃;
R⁹ is selected from: 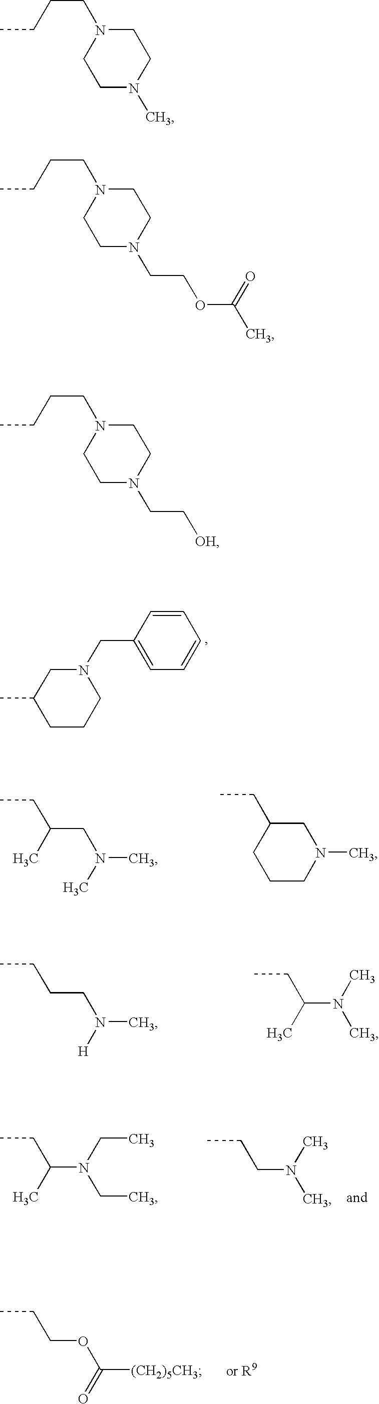
has the formula: 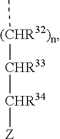
wherein n is 0 or 1, Z is NR³⁵R³⁶ or OR³⁷; each of R³², R³³, R³⁴, R³⁵, R³⁶, and R³⁷ is, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, acyl, or C₁₋₇ heteroalkyl; or any of R³³, R³⁴, R³⁵, R³⁶, and R³⁷ can be optionally taken together with intervening carbon or non-vicinal O, S or N atoms to form one or more five- to seven-membered rings, optionally substituted by H, halogen, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, acyl, or C₁₋₇ heteroalkyl;
each of R¹, R³, R⁴, R⁵, R⁶, R⁷, and R⁸ is independently H, OH, F, OCF₃, or OCH₃; and
W is NO, 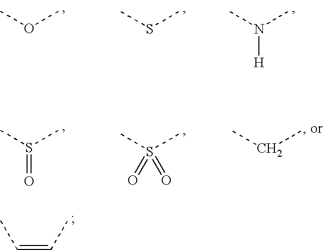
(b) a Group A antiproliferative agent; and
(c) instructions for administering said compound of formula (I) and said Group A antiproliferative agent to a patient diagnosed with or at risk of developing a neoplasm,
with the proviso that said kit does not include a bis-benzimidazole compound, an endo-exonuclease inhibitor, a PRL phosphatase inhibitor, or a PTP1B inhibitor.

29. The kit of claim 28, wherein said compound of formula (I) and said Group A antiproliferative agent are formulated separately and in individual dosage amounts.

30. The kit of claim 28, wherein said compound of formula (I) and said Group A antiproliferative agent are formulated together and in individual dosage amounts.

31. A kit comprising:
(a) a compound having the formula (I): 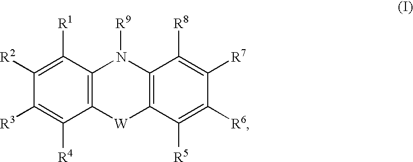
or a pharmaceutically acceptable salt thereof,
wherein R² is selected from CF₃, halogen, OCH₃, COCH₃, CN, OCF₃, COCH₂CH₃, CO(CH₂)₂CH₃, and SCH₂CH₃;
R⁹ is selected from: 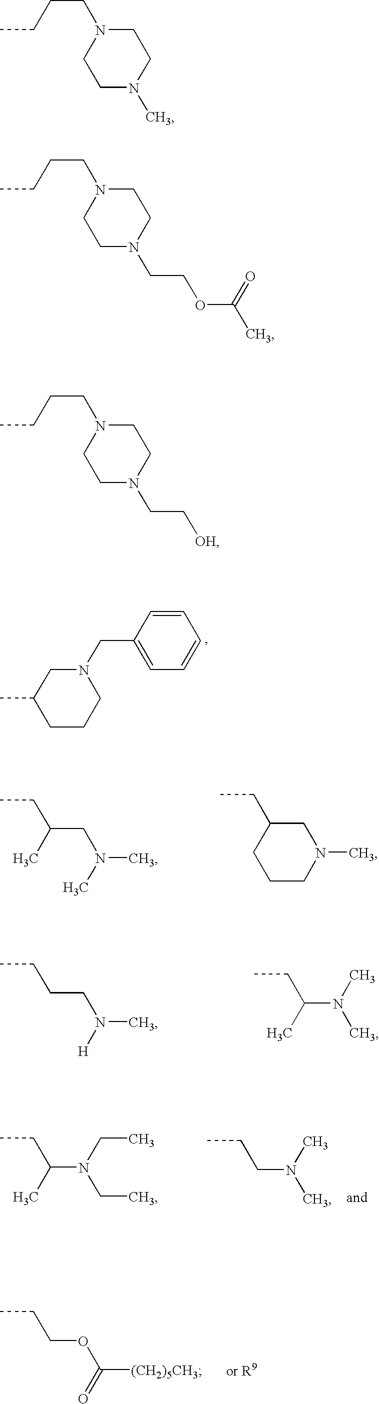
has the formula: 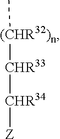
wherein n is 0 or 1, Z is NR³⁵R³⁶ or OR³⁷; each of R³², R³³, R³⁴, R³⁵, R³⁶, and R³⁷ is, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, acyl, or C₁₋₇ heteroalkyl; or any of R³³, R³⁴, R³⁵, R³⁶, and R³⁷ can be optionally taken together with intervening carbon or non-vicinal O, S, or N atoms to form one or more five- to seven-membered rings, optionally substituted by H, halogen, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, acyl, or C₁₋₇ heteroalkyl;
each of R¹, R³, R⁴, R⁵, R⁶, R⁷, and R⁸ is independently H, OH, F, OCF₃, or OCH₃; and
W is selected from NO, 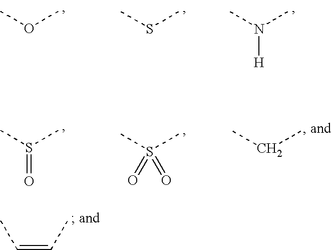
(b) instructions for administering said compound of formula (I) with a Group A antiproliferative agent to a patient diagnosed with or at risk of developing a neoplasm,
with the proviso that said kit does not include a bis-benzimidazole compound, an endo-exonuclease inhibitor, a PRL phosphatase inhibitor, or a PTP1B inhibitor.

32. The kit of claims 28 or 30, wherein said compound of formula (I) is formulated for intravenous, intramuscular, subcutaneous, buccal, inhalation, rectal, topical, or oral administration.

33. The kit of claims 28 or 30, wherein said compound of formula (I) is selected from chlorpromazine and trifluoperazine.

34. The kit of claims 28 or 30, wherein said Group A antiproliferative agent is selected from carmustine, cisplatin, etoposide, melphalan, mercaptopurine, methotrexate, mitomycin, vinblastine, paclitaxel, docetaxel, vincristine, vinorelbine, cyclophosphamide, chlorambucil, gemcitabine, capecitabine, 5-fluorouracil, fludarabine, raltitrexed, irinotecan, topotecan, doxorubicin, epirubicin, letrozole, anastrazole, formestane, exemestane, tamoxifen, toremofine, goserelin, leuporelin, bicalutamide, flutamide, nilutamide, hypericin, trastuzumab, rituximab, and combinations thereof.
